SEQUENCE LISTING (5 sequences)

SEQ ID NO: 1 - DNA, 12493 bp, Homo sapiens
  CDS (197)..(9964)
ctaccgggcggaggtgagcgcggcgccggctcctcctgcggcggactttgggtgcgacttgacgagcggtggttcgacaagtggccttgcgggccggatcgtcccagtggaagagttgtaaatttgcttctggccttcccctacggattatacctggccttcccctacggattatactcaacttactgtttagaaaatgtggcccacgagacgcctggttactatcaaaaggagcggggtcgacggtccccactttcccctgagcctcagcacctgcttgtttggaaggggtattgaatgtgacatccgtatccagcttcctgttgtgtcaaaacaacattgcaaagttgaaatccatgagcaggaggcaatattacataatttcagttccacaaatccaacacaagtaaatgggtctgttattgatgagcctgtacggctaaaacatggagatgtaataactattattgatcgttccttcaggtatgaaaatgaaagtcttcagaatggaaggaagtcaactgaatttccaagaaaaatacgtgaacaggagccagcacgtcgtgtctcaagatctagcttctcttctgaccctgatgagaaagctcaagattccaaggcctattcaaaaatcactgaaggaaaagtttcaggaaatcctcaggtacatatcaagaatgtcaaagaagacagtaccgcagatgactcaaaagacagtgttgctcagggaacaactaatgttcattcctcagaacatgctggacgtaatggcagaaatgcagctgatcccatttctggggattttaaagaaatttccagcgttaaattagtgagccgttatggagaattgaagtctgttcccactacacaatgtcttgacaatagcaaaaaaaatgaatctcccttttggaagctttatgagtcagtgaagaaagagttggatgtaaaatcacaaaaagaaaatgtcctacagtattgtagaaaatctggattacaaactgattacgcaacagagaaagaaagtgctgatggtttacagggggagacccaactgttggtctcgcgtaagtcaagaccaaaatctggtgggagcggccacgctgtggcagagcctgcttcacctgaacaagagcttgaccagaacaaggggaagggaagagacgtggagtctgttcagactcccagcaaggctgtgggcgccagctttcctctctatgagccggctaaaatgaagacccctgtacaatattcacagcaacaaaattctccacaaaaacataagaacaaagacctgtatactactggtagaagagaatctgtgaatctgggtaaaagtgaaggcttcaaggctggtgataaaactcttactcccaggaagctttcaactagaaatcgaacaccagctaaagttgaagatgcagctgactctgccactaagccagaaaatctctcttccaaaaccagaggaagtattcctacagatgtggaagttctgcctacggaaactgaaattcacaatgagccatttttaactctgtggctcactcaagttgagaggaagatccaaaaggattccctcagcaagcctgagaaattgggcactacagctggacagatgtgctctgggttacctggtcttagttcagttgatatcaacaactttggtgattccattaatgagagtgagggaatacctttgaaaagaaggcgtgtgtcctttggtgggcacctaagacctgaactatttgatgaaaacttgcctcctaatacgcctctcaaaaggggagaagccccaaccaaaagaaagtctctggtaatgcacactccacctgtcctgaagaaaatcatcaaggaacagcctcaaccatcaggaaaacaagagtcaggttcagaaatccatgtggaagtgaaggcacaaagcttggttataagccctccagctcctagtcctaggaaaactccagttgccagtgatcaacgccgtaggtcctgcaaaacagcccctgcttccagcagcaaatctcagacagaggttcctaagagaggaggagaaagagtggcaacctgccttcaaaagagagtgtctatcagccgaagtcaacatgatattttacagatgatatgttccaaaagaagaagtggtgcttcggaagcaaatctgattgttgcaaaatcatgggcagatgtagtaaaacttggtgcaaaacaaacacaaactaaagtcataaaacatggtcctcaaaggtcaatgaacaaaaggcaaagaagacctgctactccaaagaagcctgtgggcgaagttcacagtcaatttagtacaggccacgcaaactctccttgtaccataataatagggaaagctcatactgaaaaagtacatgtgcctgctcgaccctacagagtgctcaacaacttcatttccaaccaaaaaatggactttaaggaagatctttcaggaatagctgaaatgttcaagaccccagtgaaggagcaaccgcagttgacaagcacatgtcacatcgctatttcaaattcagagaatttgcttggaaaacagtttcaaggaactgattcaggagaagaacctctgctccccacctcagagagttttggaggaaatgtgttcttcagtgcacagaatgcagcaaaacagccatctgataaatgctctgcaagccctcccttaagacggcagtgtattagagaaaatggaaacgtagcaaaaacgcccaggaacacctacaaaatgacttctctggagacaaaaacttcagatactgagacagagccttcaaaaacagtatccactgtaaacaggtcaggaaggtctacagagttcaggaatatacagaagctacctgtggaaagtaagagtgaagaaacaaatacagaaattgttgagtgcatcctaaaaagaggtcagaaggcaacactactacaacaaaggagagaaggagagatgaaggaaatagaaagaccttttgagacatataaggaaaatattgaattaaaagaaaacgatgaaaagatgaaagcaatgaagagatcaagaacttgggggcagaaatgtgcaccaatgtctgacctgacagacctcaagagcttgcctgatacagaactcatgaaagacacggcacgtggccagaatctcctccaaacccaagatcatgccaaggcaccaaagagtgagaaaggcaaaatcactaaaatgccctgccagtcattacaaccagaaccaataaacaccccaacacacacaaaacaacagttgaaggcatccctggggaaagtaggtgtgaaagaagagctcctagcagtcggcaagttcacacggacgtcaggggagaccacgcacacgcacagagagccagcaggagatggcaagagcatcagaacgtttaaggagtctccaaagcagatcctggacccagcagcccgtgtaactggaatgaagaagtggccaagaacgcctaaggaagaggcccagtcactagaagacctggctggcttcaaagagctcttccagacaccaggtccctctgaggaatcaatgactgatgagaaaactaccaaaatagcctgcaaatctccaccaccagaatcagtggacactccaacaagcacaaagcaatggcctaagagaagtctcaggaaagcagatgtagaggaagaattcttagcactcaggaaactaacaccatcagcagggaaagccatgcttacgcccaaaccagcaggaggtgatgagaaagacattaaagcatttatgggaactccagtgcagaaactggacctggcaggaactttacctggcagcaaaagacagctacagactcctaaggaaaaggcccaggctctagaagacctggctggctttaaagagctcttccagactcctggtcacaccgaggaattagtggctgctggtaaaaccactaaaataccctgcgactctccacagtcagacccagtggacaccccaacaagcacaaagcaacgacccaagagaagtatcaggaaagcagatgtagagggagaactcttagcgtgcaggaatctaatgccatcagcaggcaaagccatgcacacgcctaaaccatcagtaggtgaagagaaagacatcatcatatttgtgggaactccagtgcagaaactggacctgacagagaacttaaccggcagcaagagacggccacaaactcctaaggaagaggcccaggctctggaagacctgactggctttaaagagctcttccagacccctggtcatactgaagaagcagtggctgctggcaaaactactaaaatgccctgcgaatcttctccaccagaatcagcagacaccccaacaagcacaagaaggcagcccaagacacctttggagaaaagggacgtacagaaggagctctcagccctgaagaagctcacacagacatcaggggaaaccacacacacagataaagtaccaggaggtgaggataaaagcatcaacgcgtttagggaaactgcaaaacagaaactggacccagcagcaagtgtaactggtagcaagaggcacccaaaaactaaggaaaaggcccaacccctagaagacctggctggctggaaagagctcttccagacaccagtatgcactgacaagcccacgactcacgagaaaactaccaaaatagcctgcagatcacaaccagacccagtggacacaccaacaagctccaagccacagtccaagagaagtctcaggaaagtggacgtagaagaagaattcttcgcactcaggaaacgaacaccatcagcaggcaaagccatgcacacacccaaaccagcagtaagtggtgagaaaaacatctacgcatttatgggaactccagtgcagaaactggacctgacagagaacttaactggcagcaagagacggctacaaactcctaaggaaaaggcccaggctctagaagacctggctggctttaaagagctcttccagacacgaggtcacactgaggaatcaatgactaacgataaaactgccaaagtagcctgcaaatcttcacaaccagacctagacaaaaacccagcaagctccaagcgacggctcaagacatccctggggaaagtgggcgtgaaagaagagctcctagcagttggcaagctcacacagacatcaggagagactacacacacacacacagagccaacaggagatggtaagagcatgaaagcatttatggagtctccaaagcagatcttagactcagcagcaagtctaactggcagcaagaggcagctgagaactcctaagggaaagtctgaagtccctgaagacctggccggcttcatcgagctcttccagacaccaagtcacactaaggaatcaatgactaatgaaaaaactaccaaagtatcctacagagcttcacagccagacctagtggacaccccaacaagctccaagccacagcccaagagaagtctcaggaaagcagacactgaagaagaatttttagcatttaggaaacaaacgccatcagcaggcaaagccatgcacacacccaaaccagcagtaggtgaagagaaagacatcaacacgtttttgggaactccagtgcagaaactggaccagccaggaaatttacctggcagcaatagacggctacaaactcgtaaggaaaaggcccaggctctagaagaactgactggcttcagagagcttttccagacaccatgcactgataaccccacagctgatgagaaaactaccaaaaaaatactctgcaaatctccgcaatcagacccagcggacaccccaacaaacacaaagcaacggcccaagagaagcctcaagaaagcagacgtagaggaagaatttttagcattcaggaaactaacaccatcagcaggcaaagccatgcacacgcctaaagcagcagtaggtgaagagaaagacatcaacacatttgtggggactccagtggagaaactggacctgctaggaaatttacctggcagcaagagacggccacaaactcctaaagaaaaggccaaggctctagaagatctggctggcttcaaagagctcttccagacaccaggtcacactgaggaatcaatgaccgatgacaaaatcacagaagtatcctgcaaatctccacaaccagacccagtcaaaaccccaacaagctccaagcaacgactcaagatatccttggggaaagtaggtgtgaaagaagaggtcctaccagtcggcaagctcacacagacgtcagggaagaccacacagacacacagagagacagcaggagatggaaagagcatcaaagcgtttaaggaatctgcaaagcagatgctggacccagcaaactatggaactgggatggagaggtggccaagaacacctaaggaagaggcccaatcactagaagacctggccggcttcaaagagctcttccagacaccagaccacactgaggaatcaacaactgatgacaaaactaccaaaatagcctgcaaatctccaccaccagaatcaatggacactccaacaagcacaaggaggcggcccaaaacacctttggggaaaagggatatagtggaagagctctcagccctgaagcagctcacacagaccacacacacagacaaagtaccaggagatgaggataaaggcatcaacgtgttcagggaaactgcaaaacagaaactggacccagcagcaagtgtaactggtagcaagaggcagccaagaactcctaagggaaaagcccaacccctagaagacttggctggcttgaaagagctcttccagacaccagtatgcactgacaagcccacgactcacgagaaaactaccaaaatagcctgcagatctccacaaccagacccagtgggtaccccaacaatcttcaagccacagtccaagagaagtctcaggaaagcagacgtagaggaagaatccttagcactcaggaaacgaacaccatcagtagggaaagctatggacacacccaaaccagcaggaggtgatgagaaagacatgaaagcatttatgggaactccagtgcagaaattggacctgccaggaaatttacctggcagcaaaagatggccacaaactcctaaggaaaaggcccaggctctagaagacctggctggcttcaaagagctcttccagacaccaggcactgacaagcccacgactgatgagaaaactaccaaaatagcctgcaaatctccacaaccagacccagtggacaccccagcaagcacaaagcaacggcccaagagaaacctcaggaaagcagacgtagaggaagaatttttagcactcaggaaacgaacaccatcagcaggcaaagccatggacaccccaaaaccagcagtaagtgatgagaaaaatatcaacacatttgtggaaactccagtgcagaaactggacctgctaggaaatttacctggcagcaagagacagccacagactcctaaggaaaaggctgaggctctagaggacctggttggcttcaaagaactcttccagacaccaggtcacactgaggaatcaatgactgatgacaaaatcacagaagtatcctgtaaatctccacagccagagtcattcaaaacctcaagaagctccaagcaaaggctcaagatacccctggtgaaagtggacatgaaagaagagcccctagcagtcagcaagctcacacggacatcaggggagactacgcaaacacacacagagccaacaggagatagtaagagcatcaaagcgtttaaggagtctccaaagcagatcctggacccagcagcaagtgtaactggtagcaggaggcagctgagaactcgtaaggaaaaggcccgtgctctagaagacctggttgacttcaaagagctcttctcagcaccaggtcacactgaagagtcaatgactattgacaaaaacacaaaaattccctgcaaatctcccccaccagaactaacagacactgccacgagcacaaagagatgccccaagacacgtcccaggaaagaagtaaaagaggagctctcagcagttgagaggctcacgcaaacatcagggcaaagcacacacacacacaaagaaccagcaagcggtgatgagggcatcaaagtattgaagcaacgtgcaaagaagaaaccaaacccagtagaagaggaacccagcaggagaaggccaagagcacctaaggaaaaggcccaacccctggaagacctggccggcttcacagagctctctgaaacatcaggtcacactcaggaatcactgactgctggcaaagccactaaaataccctgcgaatctcccccactagaagtggtagacaccacagcaagcacaaagaggcatctcaggacacgtgtgcagaaggtacaagtaaaagaagagccttcagcagtcaagttcacacaaacatcaggggaaaccacggatgcagacaaagaaccagcaggtgaagataaaggcatcaaagcattgaaggaatctgcaaaacagacaccggctccagcagcaagtgtaactggcagcaggagacggccaagagcacccagggaaagtgcccaagccatagaagacctagctggcttcaaagacccagcagcaggtcacactgaagaatcaatgactgatgacaaaaccactaaaataccctgcaaatcatcaccagaactagaagacaccgcaacaagctcaaagagacggcccaggacacgtgcccagaaagtagaagtgaaggaggagctgttagcagttggcaagctcacacaaacctcaggggagaccacgcacaccgacaaagagccggtaggtgagggcaaaggcacgaaagcatttaagcaacctgcaaagcggaacgtggacgcagaagatgtaattggcagcaggagacagccaagagcacctaaggaaaaggcccaacccctggaagacctggccagcttccaagagctctctcaaacaccaggccacactgaggaactggcaaatggtgctgctgatagctttacaagcgctccaaagcaaacacctgacagtggaaaacctctaaaaatatccagaagagttcttcgggcccctaaagtagaacccgtgggagacgtggtaagcaccagagaccctgtaaaatcacaaagcaaaagcaacacttccctgcccccactgcccttcaagaggggaggtggcaaagatggaagcgtcacgggaaccaagaggctgcgctgcatgccagcaccagaggaaattgtggaggagctgccagccagcaagaagcagagggttgctcccagggcaagaggcaaatcatccgaacccgtggtcatcatgaagagaagtttgaggacttctgcaaaaagaattgaacctgcggaagagctgaacagcaacgacatgaaaaccaacaaagaggaacacaaattacaagactcggtccctgaaaataagggaatatccctgcgctccagacgccaagataagactgaggcagaacagcaaataactgaggtctttgtattagcagaaagaatagaaataaacagaaatgaaaagaagcccatgaagacctccccagagatggacattcagaatccagatgatggagcccggaaacccatacctagagacaaagtcactgagaacaaaaggtgcttgaggtctgctagacagaatgagagctcccagcctaaggtggcagaggagagcggagggcagaagagtgcgaaggttctcatgcagaatcagaaagggaaaggagaagcaggaaattcagactccatgtgcctgagatcaagaaagacaaaaagccagcctgcagcaagcactttggagagcaaatctgtgcagagagtaacgcggagtgtcaagaggtgtgcagaaaatccaaagaaggctgaggacaatgtgtgtgtcaagaaaataacaaccagaagtcatagggacagtgaagatatttgacagaaaaatcgaactgggaaaaatataataaagttagttttgtgataagttctagtgcagtttttgtcataaattacaagtgaattctgtaagtaaggctgtcagtctgcttaagggaagaaaactttggatttgctgggtctgaatcggcttcataaactccactgggagcactgctgggctcctggactgagaatagttgaacaccgggggctttgtgaaggagtctgggccaaggtttgccctcagctttgcagaatgaagccttgaggtctgtcaccacccacagccaccctacagcagccttaactgtgacacttgccacactgtgtcgtcgtttgtttgcctatgttctccagggcacggtggcaggaacaactatcctcgtctgtcccaacactgagcaggcactcggtaaacacgaatgaatggataagcgcacggatgaatggagcttacaagatctgtctttccaatggccgggggcatttggtccccaaattaaggctattggacatctgcacaggacagtcctatttttgatgtcctttcctttctgaaaataaagttttgtgctttggagaatgactcgtgagcacatctttagggaccaagagtgactttctgtaaggagtgactcgtggcttgccttggtctcttgggaatacttttctaactagggttgctctcacctgagacattctccacccgcggaatctcagggtcccaggctgtgggccatcacgacctcaaactggctcctaatctccagctttcctgtcattgaaagcttcggaagtttactggctctgctcccgcctgttttctttctgactctatctggcagcccgatgccacccagtacaggaagtgacaccagtactctgtaaagcatcatcatccttggagagactgagcactcagcaccttcagccacgatttcaggatcgcttccttgtgagccgctgcctccgaaatctcctttgaagcccagacatctttctccagcttcagacttgtagatataactcgttcatcttcatttactttccactttgccccctgtcctctctgtgttccccaaatcagagaatagcccgccatcccccagatcacctgtctggattcctccccattcacccaccttgccaggtgcaggtgaggatggtgcaccagacagggtagctgtcccccaaaatgtgccctgtgcgggcagtgccctgtctccacgtttgtttccccagtgtctggcggggagccaggtgacatcataaatacttgctgaatgaatgcagaaatcagcggtactgacttgtactatattggctgccatgatagggttctcacagcgtcatccatgatcgtaagggagaatgacattctgcttgagggagggaatagaaaggggcagggaggggacatctgagggcttcacagggctgcaaagggtacagggattgcaccagggcagaacaggggagggtgttcaaggaagagtggctcttagcagaggcactttggaaggtgtgaggcataaatgcttccttctacgtaggccaacctcaaaactttcagtaggaatgttgctatgatcaagttgttctaacactttagacttagtagtaattatgaacctcacatagaaaaatttcatccagccatatgcctgtggagtggaatattctgtttagtagaaaaatcctttagagttcagctctaaccagaaatcttgctgaagtatgtcagcaccttttctcaccctggtaagtacagtatttcaagagcacgctaagggtggttttcattttacagggctgttgatgatgggttaaaaatgttcatttaagggctacccccgtgtttaatagatgaacaccacttctacacaaccctccttggtactgggggagggagagatctgacaaatactgcccattcccctaggctgactggatttgagaacaaatacccacccatttccaccatggtatggtaacttctctgagcttcagtttccaagtgaatttccatgtaataggacattcccattaaatacaagctgtttttactttttcgcctcccagggcctgtgcgatctggtcccccagcctctcttgggctttcttacactaactctgtacctaccatctcctgcctcccttaggcaggcacctccaaccaccacacactccctgctgttttccctgcctggaactttcccaccagccccaccaagatcatttcatccagtcctgagctcagcttaagggaggcttcttgcctgtgggttccctcacccccatgcctgtcctccaggctggggcaggttcttagtttgcctggaattgttctgtacctctttgtagcacgtagtgttgtgaaactaagccactaattgagtttctggctcccctcctggggttgtaagttttgttcattcatgagggccgactgtatttcctggttactgtatcccagtgaccagccacaggagatgtccaataaagtatgtgatgaaatggtctt

SEQ ID NO: 2 - protein, 3256 aa, Homo sapiens
MWPTRRLVTIKRSGVDGPHFPLSLSTCLFGRGIECDIRIQLPVVSKQHCKVEIHEQEAILHNFSSTNPTQVNGSVIDEPVRLKHGDVITIIDRSFRYENESLQNGRKSTEFPRKIREQEPARRVSRSSFSSDPDEKAQDSKAYSKITEGKVSGNPQVHIKNVKEDSTADDSKDSVAQGTTNVHSSEHAGRNGRNAADPISGDFKEISSVKLVSRYGELKSVPTTQCLDNSKKNESPFWKLYESVKKELDVKSQKENVLQYCRKSGLQTDYATEKESADGLQGETQLLVSRKSRPKSGGSGHAVAEPASPEQELDQNKGKGRDVESVQTPSKAVGASFPLYEPAKMKTPVQYSQQQNSPQKHKNKDLYTTGRRESVNLGKSEGFKAGDKTLTPRKLSTRNRTPAKVEDAADSATKPENLSSKTRGSIPTDVEVLPTETEIHNEPFLTLWLTQVERKIQKDSLSKPEKLGTTAGQMCSGLPGLSSVDINNFGDSINESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGERVATCLQKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTVNRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGWKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDLDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTADEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPVCTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRNVDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQDKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKITTRSHRDSEDI

SEQ ID NO: 3 - DNA, 23 bp, Artificial sequence
  Description of the artificial sequence start-2-anti Synthetic oligonucleotide
accaggcgtctcgtgggccacat

SEQ ID NO: 4 - DNA, 23 bp, Artificial sequence
  Description of the artificial sequence start-2-sense Synthetic oligonucleotide
atgtggcccacgagacgcctggt

SEQ ID NO: 5 - DNA, 25 bp, Artificial sequence
  Description of the artificial sequence missense Synthetic oligonucleotide
agtactcagtaacgcctacggtaag

(19) United States
(12) Patent Application Publication
(10) Pub. No.: US 2004/0242519 A1
(43) Pub. Date: Dec. 2, 2004

(54) Antisense oligonucleotides for treatment of proliferating cells

(76) Inventors: Hans-Dieter Flad, Borstel (DE); Johannes Gerdes, Feldhorst (DE); Andreas Bohle, Gross Gronau (DE); Irina Enrchen-Deinert, Steinburg (DE)

Correspondence Address: MANELLI DENISON & SELTER, 2000 M STREET NW SUITE 700, WASHINGTON, DC, 20036-3307, US

(21) Appl. No.: 10/739,946

(22) Filed: Dec. 19, 2003

Related U.S. Application Data
(63) Continuation of application No. 09/700,906, filed on Feb. 26, 2001, now abandoned.
Application No. 09/700,906 is a 371 of international application No. PCT/EP99/03451, filed on May 20, 1999.

(30) Foreign Application Priority Data
May 22, 1998 (DE) 198 22 954.2

(51) Int. Cl. A61K 48/00

(52) U.S. Cl. 514/44

(57) ABSTRACT

The invention relates to oligoribo- and oligodeoxyribonucleotides which are suitable for treating pathological conditions accompanied by increased cell proliferation. The oligoribo- and oligodeoxyribonualeotides are characterized in that they are able to hybridise with the mRNA which codes for the cell cycle-associated protein Ki-67.

The invention relates to oligoribo- and oligodeoxyribonucleotides which are suitable for treating pathological conditions accompanied by an increased cell proliferation.

Nucleic acid fragments of which the sequence is complementary to the coding or “sense” strand of DNA or a messenger RNA (mRNA) and which are therefore capable of binding specifically to these complementary target sequences (hybridizing) are called antisense oligonucleotides. Selective influencing of cell processes is possible by this means. Antisense oligonucleotides have found interest as tools in research and as potential agents for antiviral and tumour therapy (E. Uhlmann, A. Peyman, Chemical Reviews, 90 (1990) 544-584; S. Agrawal, TIBTECH 10 (1992) 152-158) and in some cases have already reached the stage of clinical research (M. D. Matteucci, R. W. Wagner, Nature 384 (196) 20-22).

Ki-67 is a cell protein which is produced in all active phases of the cell cycle (G₁, S, G₂ and mitosis), but not during the resting phase (G₀). The resting or G₀ phase describes the state in which the dividing activity of the cell is at rest, i.e. the cells have left the active phases of the cell cycle and do not divide. Ki-67 is a human nuclear protein, expression of which is associated strictly with cell proliferation. Specific antibodies against the Ki-67 protein are used in histopathology for determination of the proportion of growing cells in human tumours (J. Gerdes, Seminars in Cancer Biology 1 (1990) 199-206).

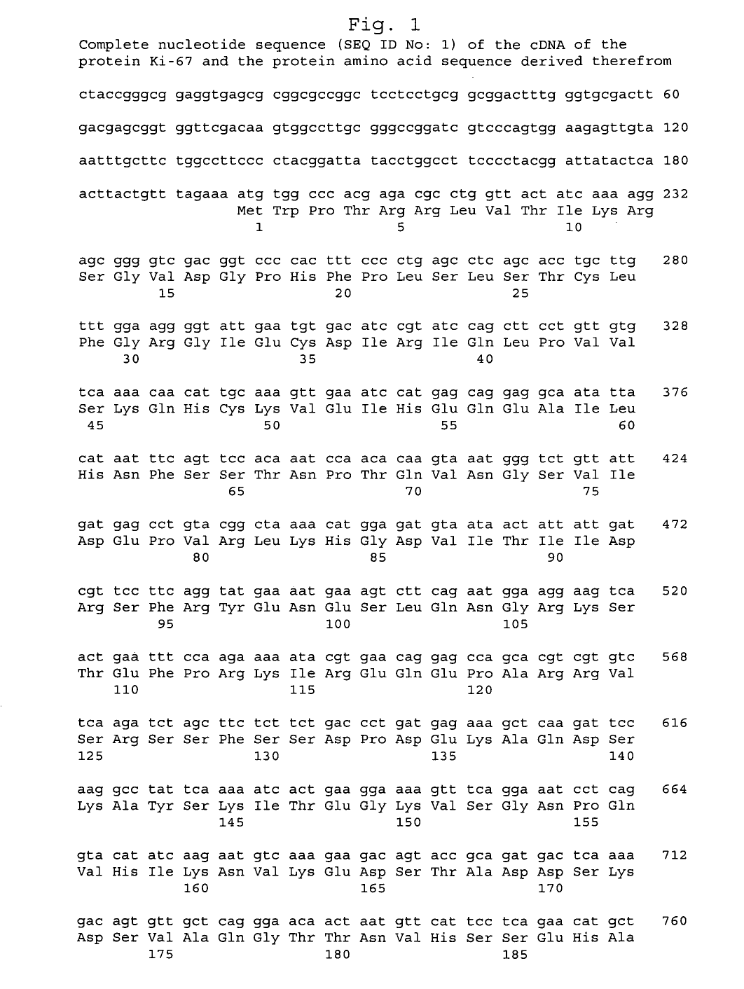

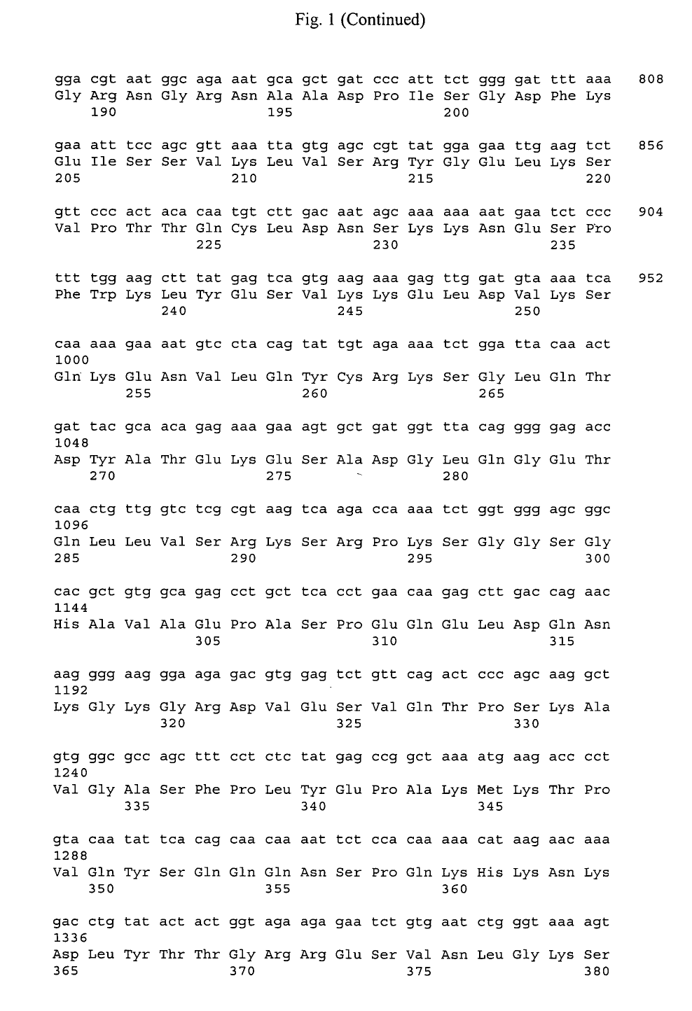

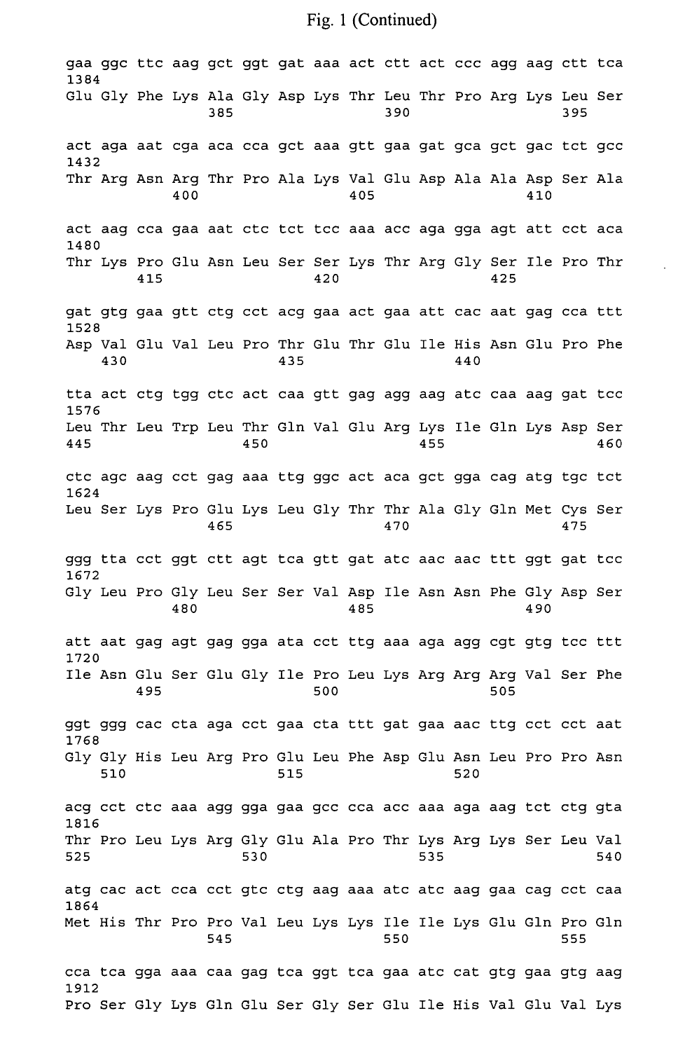

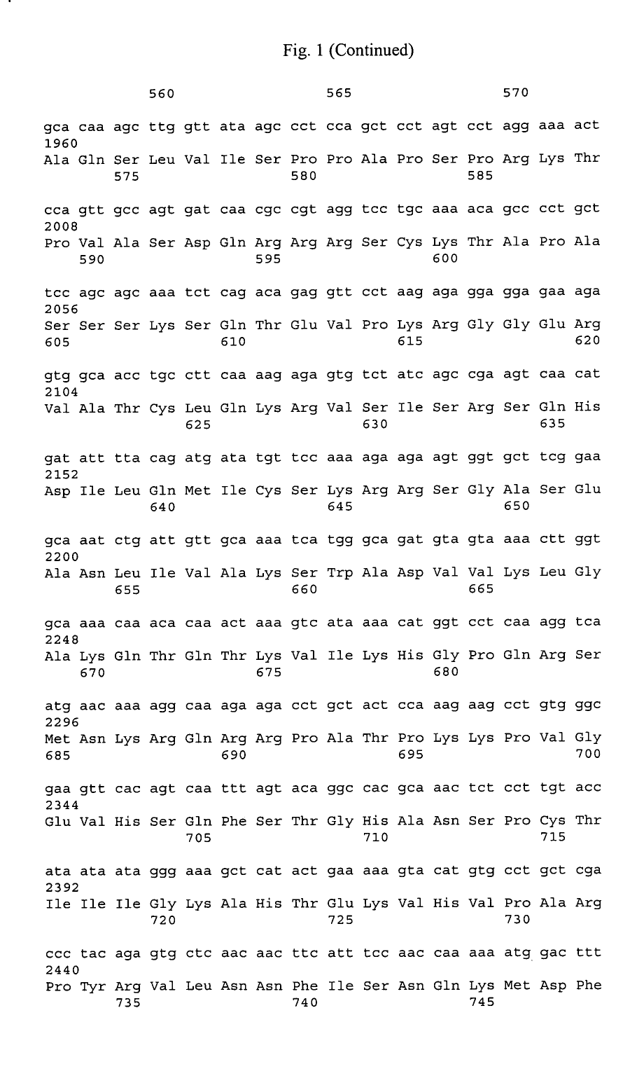

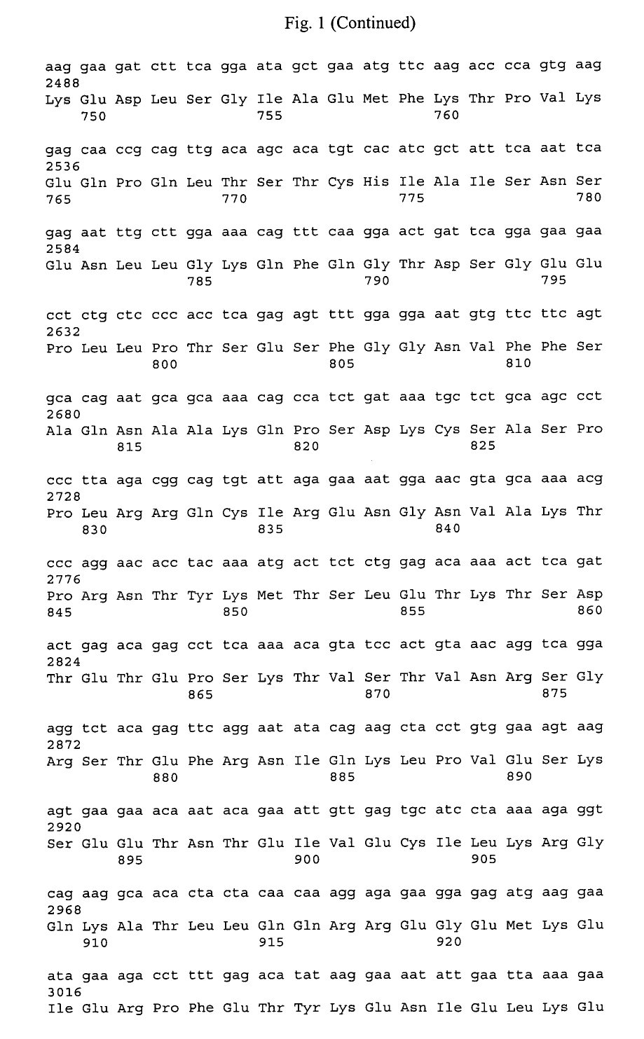

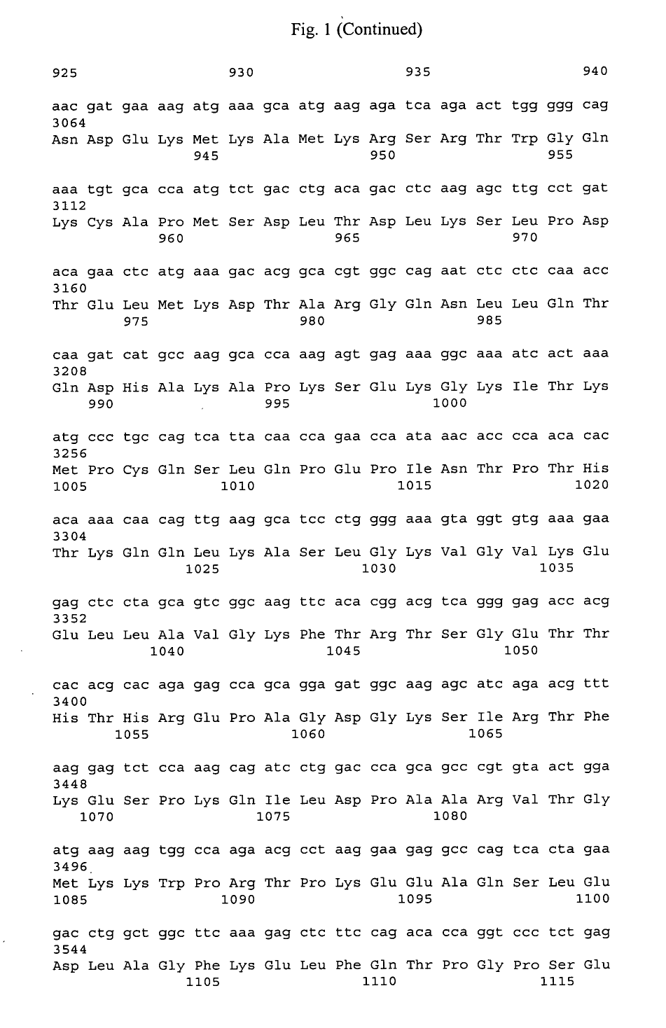

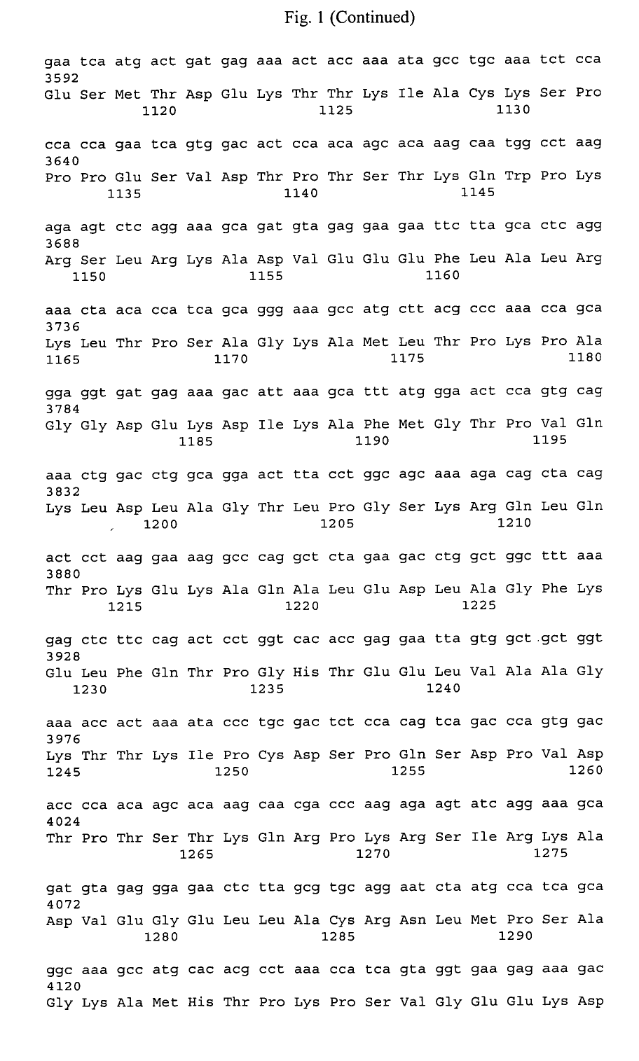

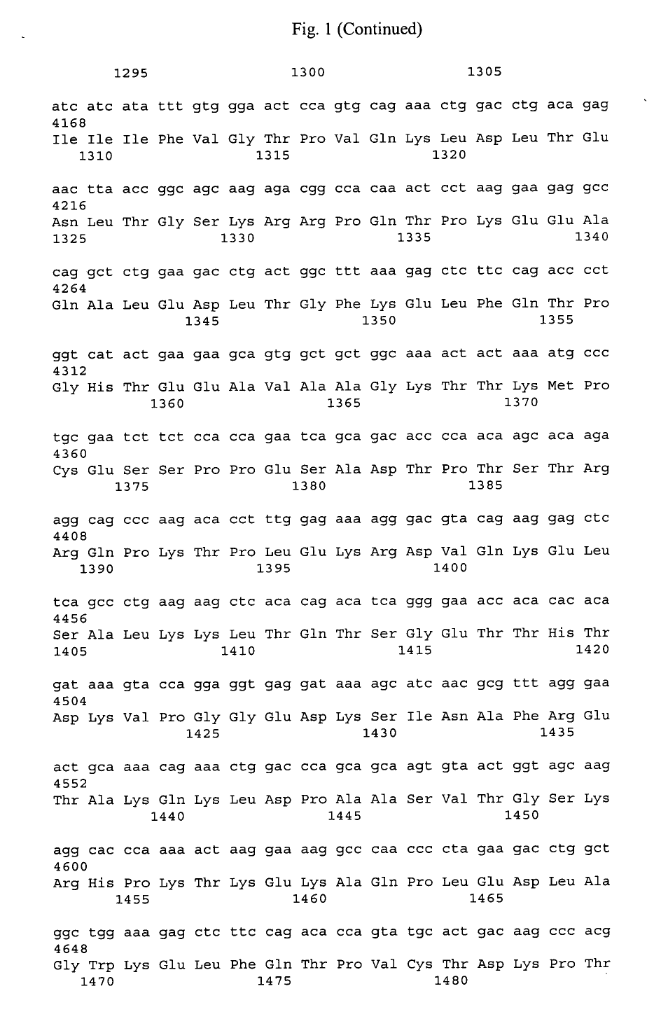

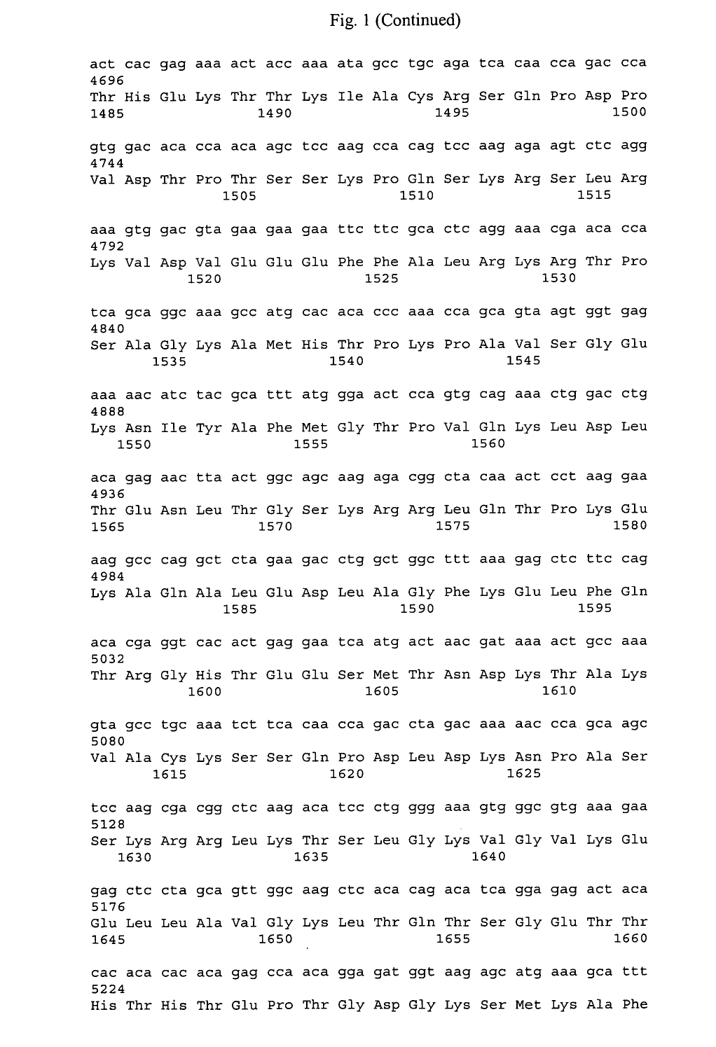

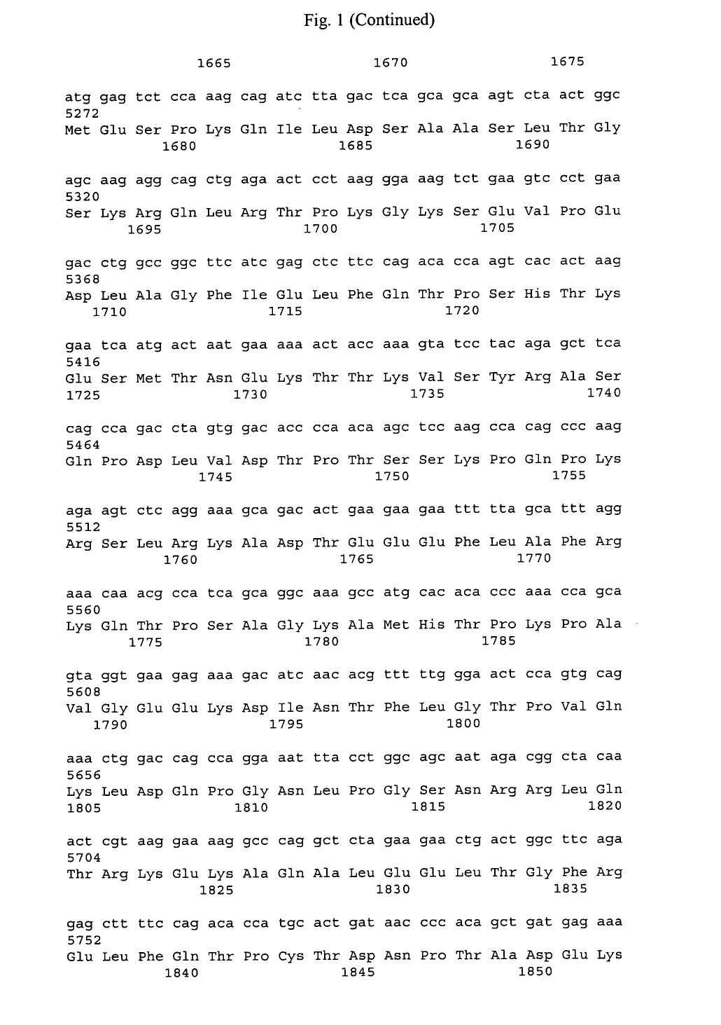

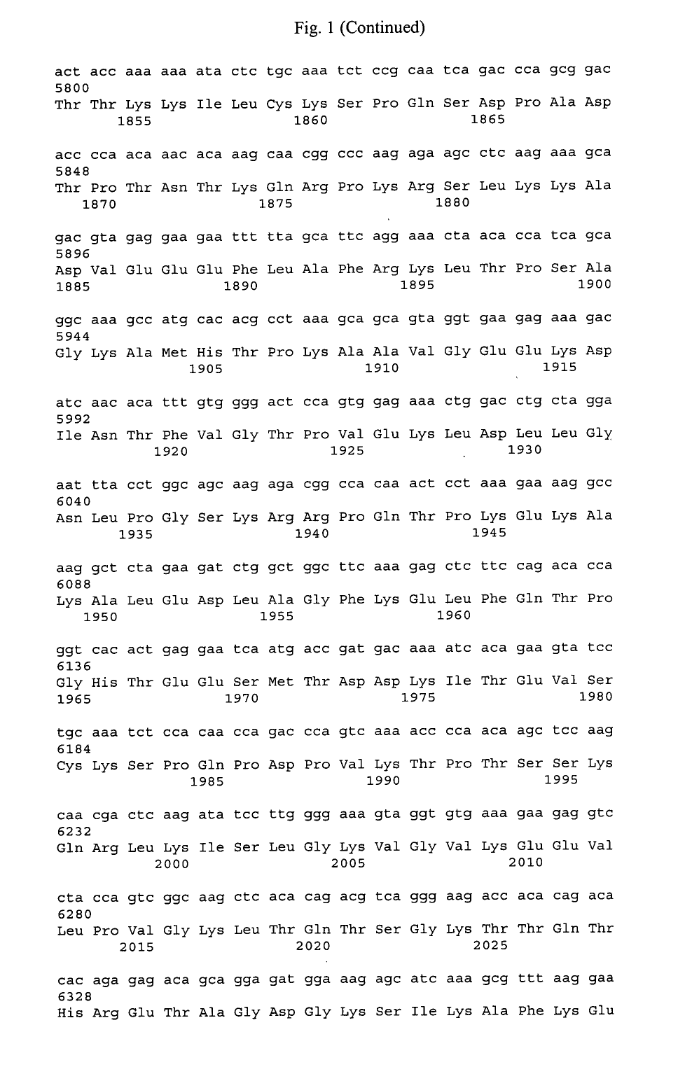

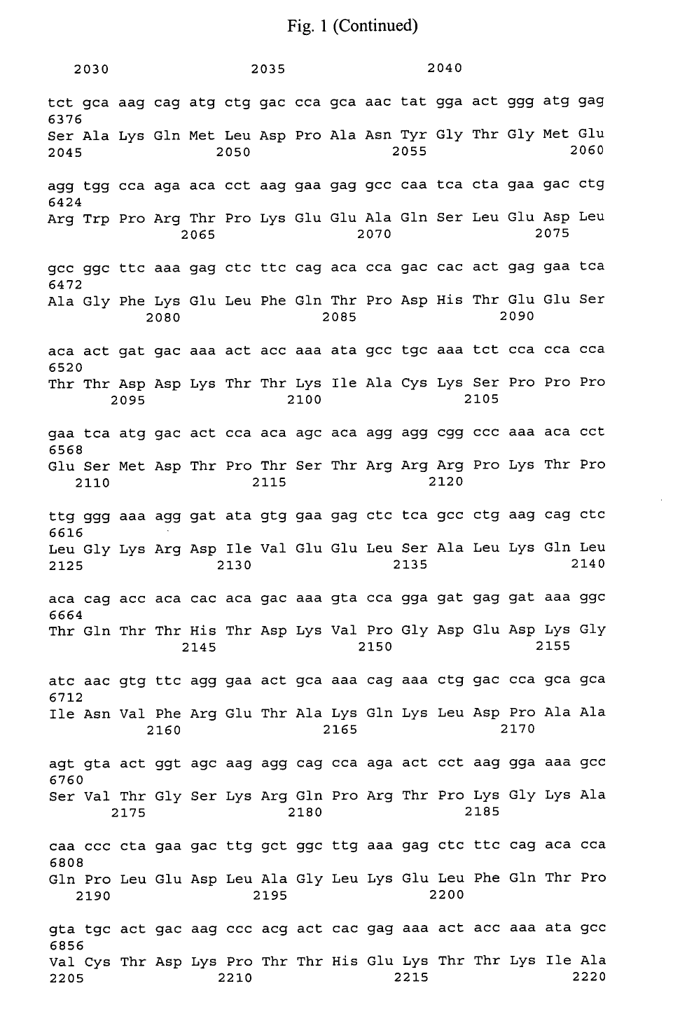

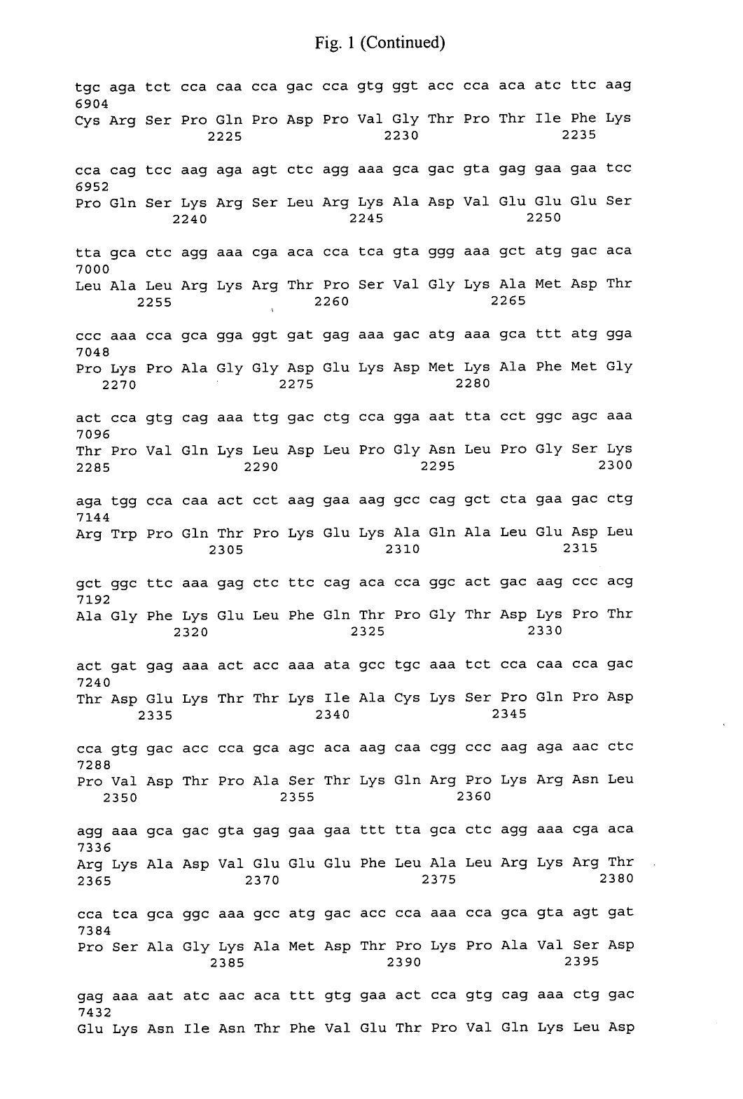

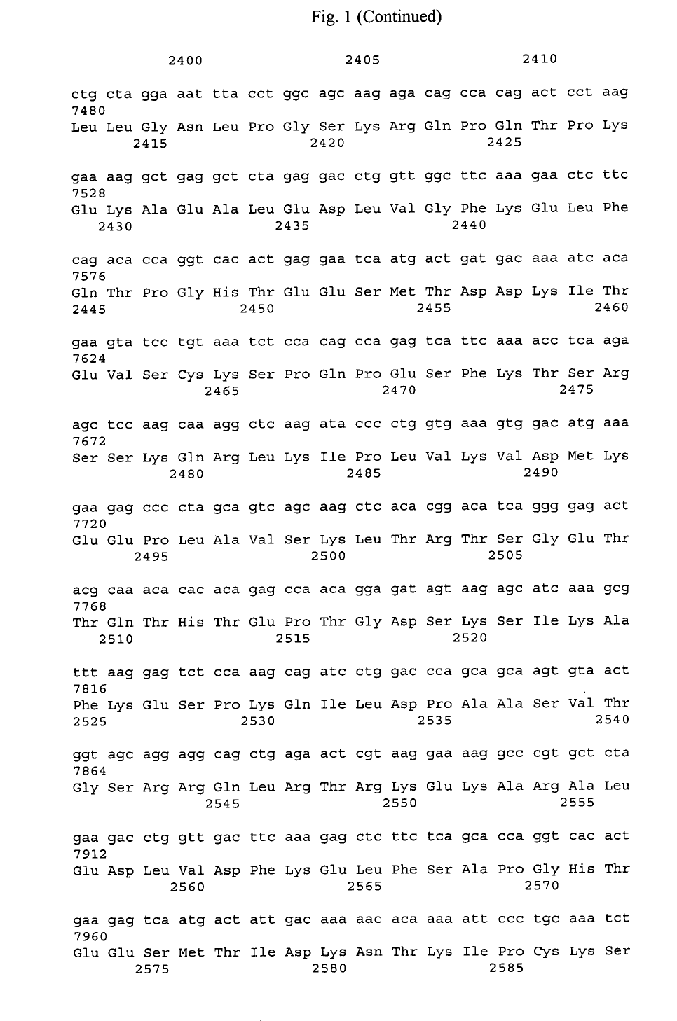

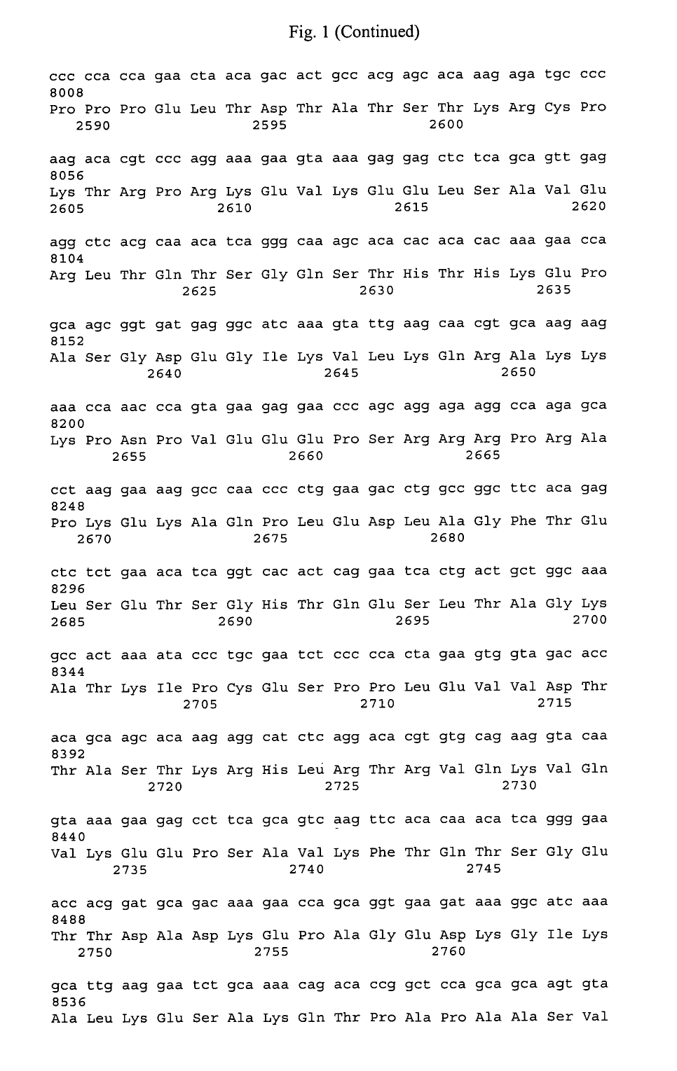

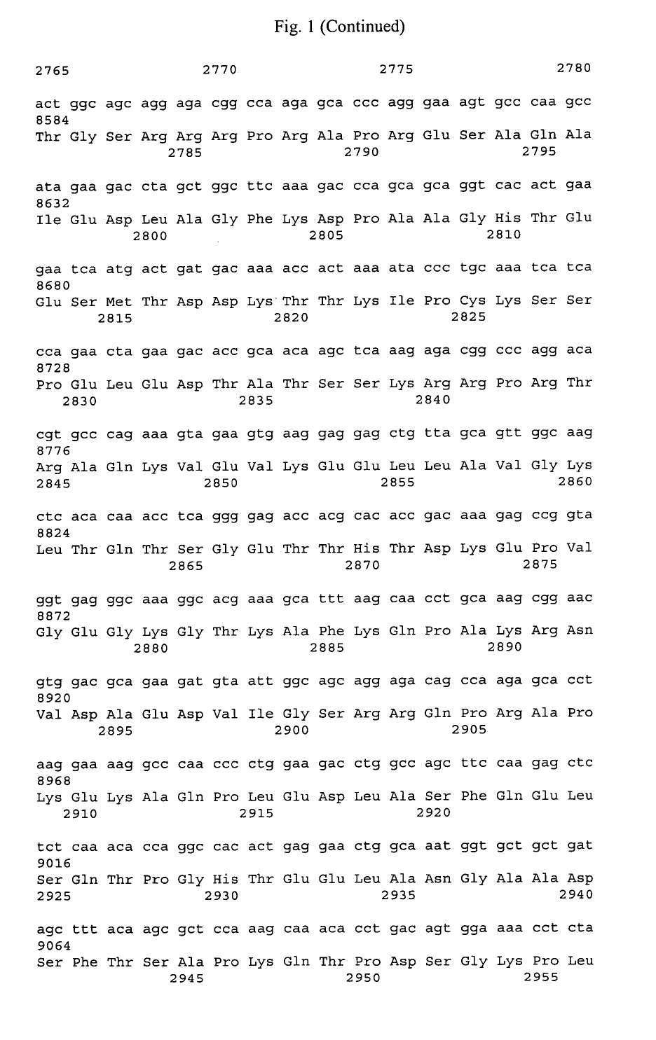

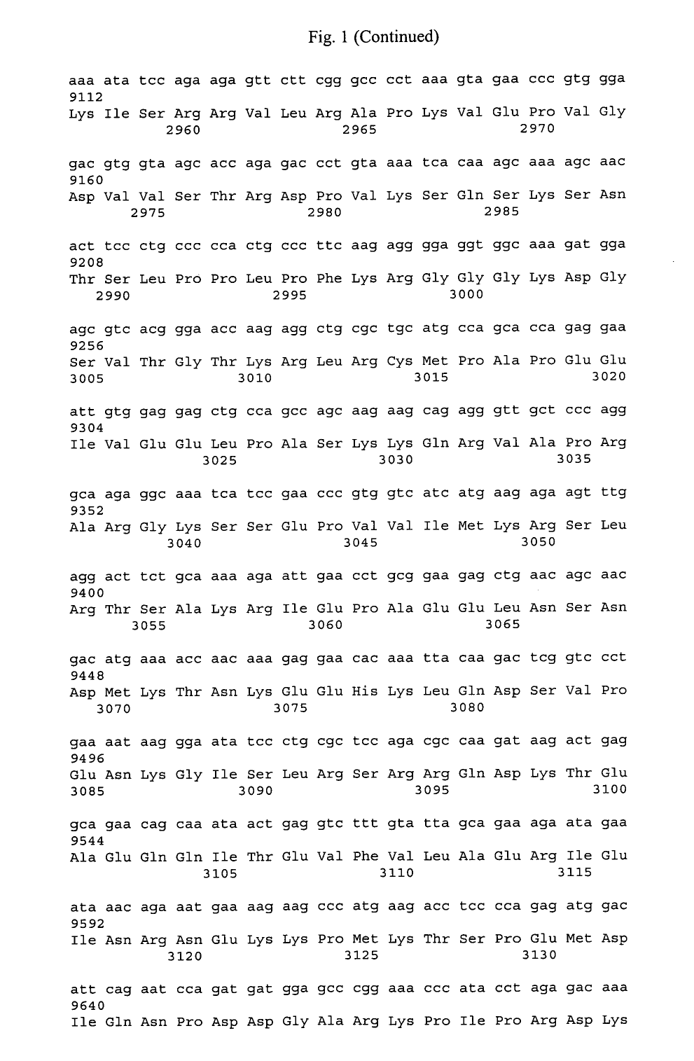

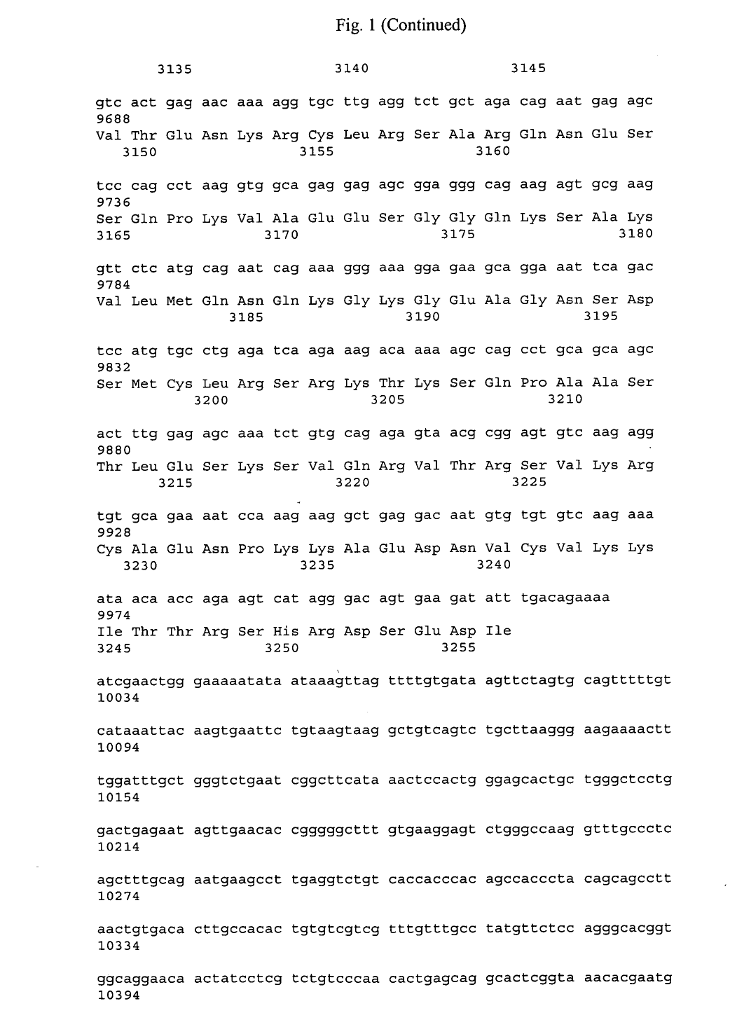

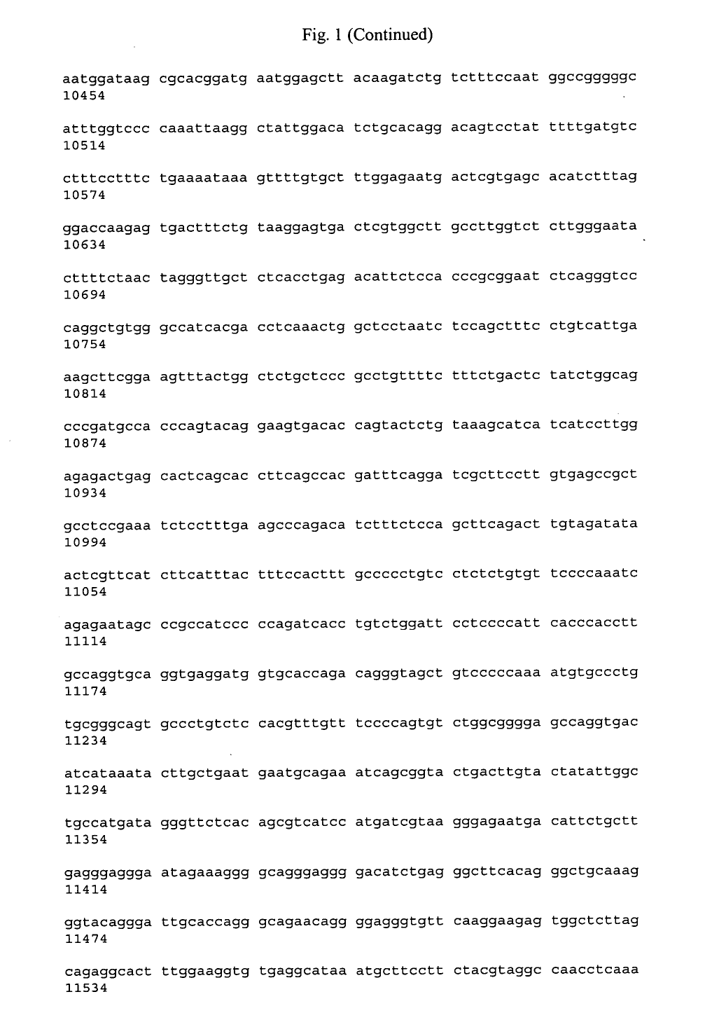

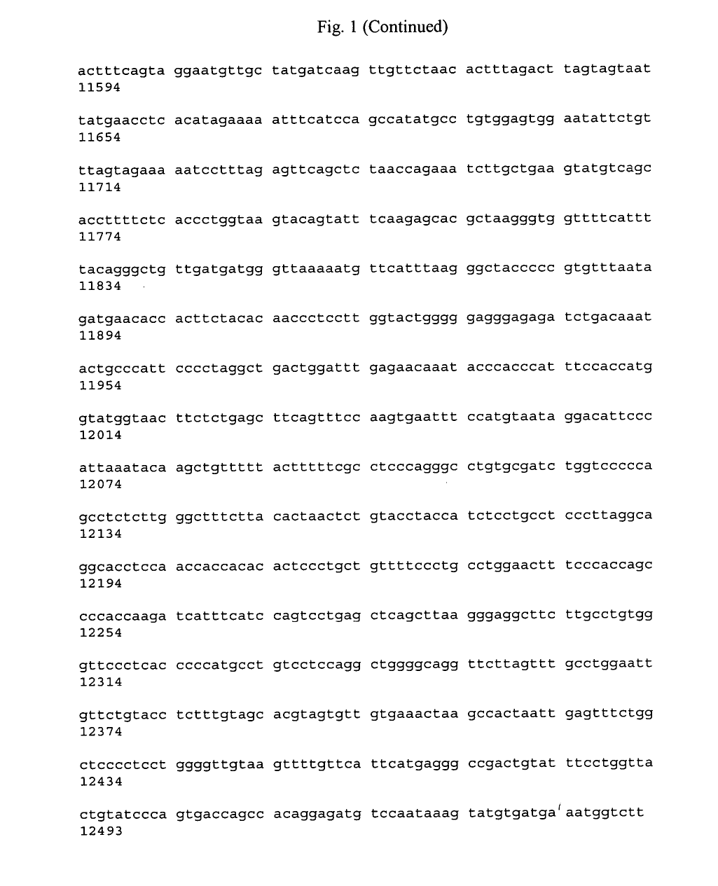

It has furthermore been found that proliferation of human IM-9 cells can be inhibited as a function of the concentration by a Ki-67 protein antisense 2′-deoxyoligonucleotide comprising 21 bases (C. Schlüter et al., The Journal of Cell Biology, 123 (1993) 513-522). The complete nucleotide sequence of the cDNA of the Ki-67 protein and the derived amino acid sequence are known (Schlüter et al., loc. cit.). FIG. 1 (SEQ ID NO 1) shows the sense strand of the Ki-67 cDNA.

The object of the present invention is to provide antisense oligonucleotides which are suitable for treating pathological conditions accompanied by an increased cell proliferation. Examples of such disease states are tumours, allergies, autoimmune diseases, cicatrization, inflammations and rheumatic diseases, as well as suppression of rejection reactions in case of transplantations.

This object has been achieved by oligoribo- or oligodeoxyribonucleotides, and physiologically acceptable salts thereof, which are capable of hybridizing with the mRNA which codes for the protein Ki-67.

It has been found that the oligoribo- or oligodeoxyribonucleotides according to the invention have a cytotoxic and not only inhibiting action on proliferating cells, such as, for example, tumour cells, and cause the death of the cells. This finding is surprising in as much as the Ki-67 protein is not detectable in non-proliferating cells and is thus evidently not necessary for survival of the cells.

Oligonucleotides which hybridise with Ki-67 mRNA at 37° C. and a physiological saline concentration are preferred.

Oligoribo- and oligodeoxyribonucleotides, and in particular oligodeoxyribonucleotides, of which the sequence is complementary to the nucleotide sequence, shown in FIG. 1 (SEQ ID NO: 1), of the sense strand of the cDNA of Ki-67, i.e. at a chain length of 10 bases has not more than 0 to 4, preferably 0 to 2, and even more preferably no mismatches, are particularly preferred.

Oligoribo- and oligodeoxyribonucleotides which hybridise with a nucleotide sequence from the 5′ region of the Ki-67 mRNA, i.e. oligoribo- or oligodeoxyribonucleotides which are complementary to the 5′ region of the sequence shown in FIG. 1, preferably to a section of the region from position 197 to 2673 or 2673 to 9962, particularly preferably 197 to 220, have furthermore proved to be particularly active.

The oligonucleotides according to the invention preferably have a chain length of 12 to 66 nucleotides, particularly preferably 17 to 46 and very particularly preferably 22 to 46 nucleotides.

The sequence (SEQ ID NO: 3):

(5′-ACC AGG CGT CTC GTG GGC CAC AT)

is very particularly preferred.

Non-modified oligonucleotides, and in particular non-modified oligoribonucleotides, are subject to nucleolytic degradation to a high degree and therefore have only a low stability and biological half-life. To improve ability to penetrate through membranes and to increase the biological half-life, the bases, sugar residues and/or phosphate residues of the oligonucleotides according to the invention are preferably modified.

Oligonucleotides in which one or more phosphate groups are replaced by phosphothioate, methylphosphonate, phosphoramidate, methylene(methylimino) (MMI) and/or guanidine groups are preferred. The structure of these groups is shown in FIG. 2. Thiolated oligonucleotides, i.e. oligonucleotides in which phosphate groups are replaced by phosphothioate groups, are particularly preferred. One or more of the phosphate groups of the oligonucleotide can be modified. In the case of partial modification, terminal groups are preferably modified, but oligonucleotides in which all the phosphate groups are modified are most preferred.

Preferred sugar modifications comprise replacement of one or more ribose residues of the oligonucleotide by hexose (FIG. 2) or by amino acids (peptide nucleic acid, PNA, FIG. 2).

Modifications of the bases comprise the use of 5-propinyl-uracyl, 5-propinylcytosine and the tricyclic cytosine analogue phenoxazine.

The synthesis of modified oligonucleotides and further suitable ways of modification are described in the literature (cf., for example, E. Uhlmann, A. Peyman, loc. cit.; M. D. Matteucci, R. W. Wagner, loc. cit.).

The oligonucleotides according to the invention can moreover be protected against degradation by exo-nucleases by terminal 3′-3′ and/or 5′-5′ internucleotide bonds (H. Seliger et al., Nucleosides & Nucleotides 10 (1-3), 469-477 (1991)).

The oligonucleotides according to the invention can furthermore additionally be substituted by groups which promote intracellular uptake, which serve in vivo or in vitro as reporter groups, and/or groups which, during hybridization of the oligoribonucleotide on the target RNA, attack the same by bonding or cleavage.

Examples of groups which promote intracellular uptake are lipophilic residues, such as alkyl residues, for example having 1 to 18 C. atoms, cholesteryl or thiocholesteryl groups (E. Uhlmann, A. Peyman, loc. cit.) or conjugates which utilise natural carrier systems, such as e.g. bile acid or peptides for the corresponding receptor (e.g. receptor-mediated endocytosis).

Examples of reporter groups are fluorescent groups (e.g. acridinyl, dansyl or fluorescinyl groups) or chemiluminescent groups, such as e.g. acridinium ester groups.

Examples of oligonucleotide conjugates which bond to and/or cleave nucleic acids are to be found in E. Uhlmann, A. Peyman, loc. cit. Conjugate partners are, inter alia, acridine, psolaren, chloroethylaminoaryl, phenanthridine, azidophenacyl, azidoproflavine, phenazine, phenanthroline/Cu, porphyrin/Fe, benzo[e]pyridoindole and EDTA/Fe (Mergny et al., Science 256 (1992) 1681).

The oligonucleotides according to the invention are prepared in a manner known per se (cf. e.g. E. Uhlmann, A. Peyman, loc. cit.). Synthesis on a solid phase with the aid of an automatic synthesis apparatus is preferred.

To prepare medicaments, the oligonucleotides according to the invention are combined with conventional carrier substances, auxiliaries and/or additives. The oligonucleotides are suitable for systemic, local, subcutaneous, intrathecal and topical use and for administration by enema. For this, they can be used as a solution in suitable solvents, preferably aqueous solutions, in the form of liposomes, as an emulsion or in solid form, for example as a powder or in microencapsulated form.

The amount of oligonucleotides in the medicaments depends on the desired use and is preferably adjusted such that an administration of 0.001 to 100 mg oligonucleotide per kg of body weight, preferably 0.001 to 10 mg/kg of body weight, particularly preferably 0.01 to 3 mg/kg of body weight is achieved. Treatment is preferably carried out by repeated use over a period of one day to 6 weeks in a dose of preferably 0.01 to 3 mg/kg per day.

The oligonucleotides according to the invention are suitable for treating pathological conditions accompanied by an increased cell proliferation, in particular for treatment of benign and malignant tumours, such as testicular tumours, lymphomas, gastric carcinomas, bladder carcinomas, mammary carcinomas, bronchial carcinomas, sarcomas, renal carcinomas and melanomas, autoimmune diseases, cicatrization, inflammations, allergies, rheumatic diseases and rejection reactions in case of transplantations.

A particular advantage of the oligonucleotides according to the invention is to be seen in that they allow treatment of tumours which are resistant to conventional chemotherapeutics. Such resistances arise either secondarily, i.e. after several administrations with non-specific cytostatics, such as, for example, vinblastin or cisplatin, or are already primarily present with certain tumours, such as, for example, renal carcinoma.

The finding that the oligonucleotides according to the invention not only inhibit the growth of cells but also have a cytotoxic action, i.e. lead to the death of the treated tumour cells, was particularly surprising. The cytotoxic action in general starts after a treatment time of about 5 to 12 days. A treatment time of some months may be necessary for complete destruction of all the proliferating cells, whereby the treatment time may be interrupted by periods of non-treatment.

The invention is explained in more detail with the following examples.

### EXAMPLE 1

### Action on the Growth of RT4 Cells in the Multicellular Spheroid Test

The action of oligonucleotides according to the invention on bladder carcinoma cells of the cell line RT4 was investigated on multicellular spheroids and compared with corresponding sense and missense strands as a control.

For this, 2′-deoxyoligonucleotides with the following sequences were prepared in a known manner (Uhlmann and Peyman, loc. cit.):

| | |
|---|---|
| start-2-anti | 5′-ACC AGG CGT CTC GTG GGC CAC AT |
| start-2-sense | 5′-ATG TGG CCC ACG AGA CGC CTG GT |
| missense | 5′-AGT ACT CAG TAA CGC CTA CGG TAA G |

Unless stated otherwise, all the oligonucleotides were employed in thiolated form, i.e. one oxygen atom of the phosphoric acid radicals was replaced by a sulphur atom.

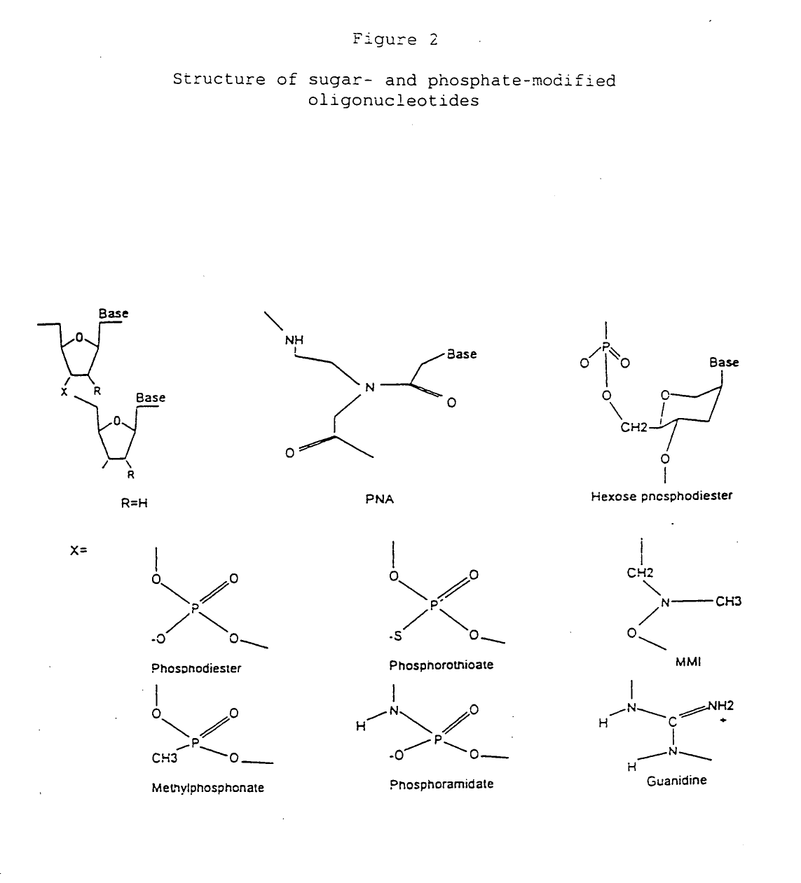

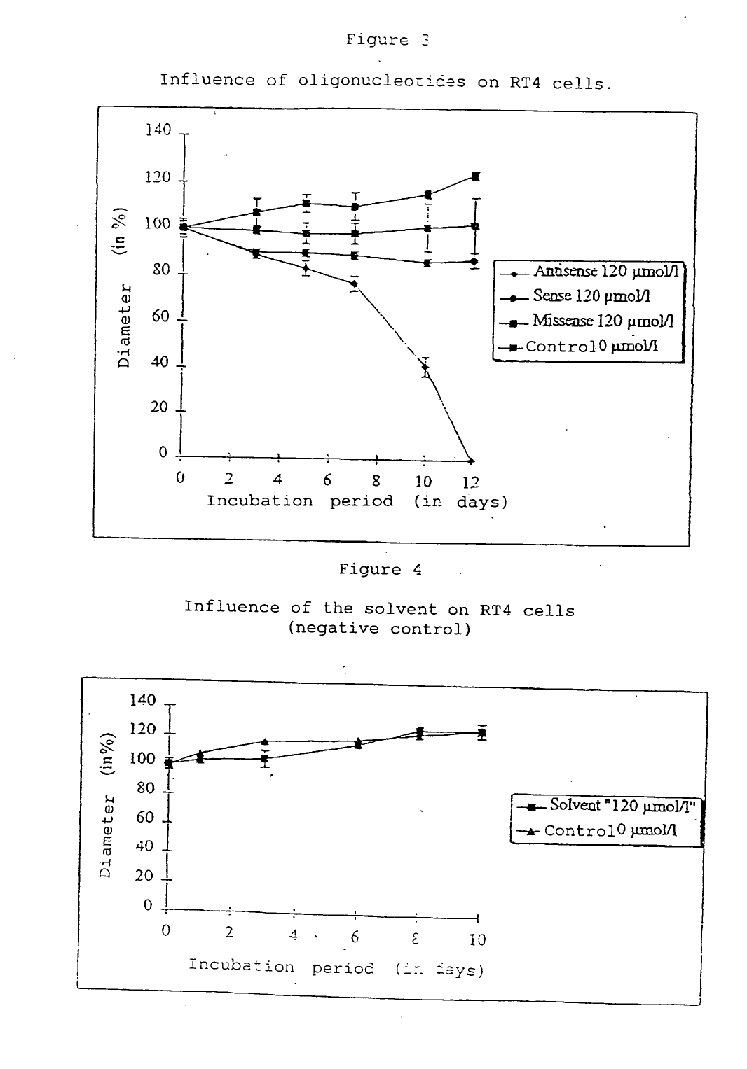

Multicellular spheroids of the cell line RT-4 (ATCC no.: HTB2) were prepared by the method of Carlsson & Yuhas (J. Carlsson and J. M. Yuhas, Liquid-overlay culture of cellular spheroids, Recent Results in Cancer Research 95; 1-23, 1984). After four days the multicellular spheroids showed a spherical morphology with a pronounced, sharp demarcation. The RT4 multicellular spheroids were then incubated in the presence of 120 μmol/l of the particular oligonucleotides in culture media at 37° C. with 5% CO₂ and the change in the spheroid diameter was measured. The oligonucleotides were introduced into the medium directly after the period of time necessary for formation of the spheroids. On the one hand a sample to which no oligonucleotides were added (control) and on the other hand the missense and sense oligonucleotide samples served as negative controls. Thereafter, the diameter of the multicellular spheroids was measured at intervals of 2 days. Three identical batches were investigated. per test and the mean was then obtained. The results are plotted as a graph in FIG. 3.

An increase in the spheroid diameter to 132% of the starting value was observed in the control, while the addition of the thiolated missense oligonucleotide caused a stop in growth. The addition of the sense oligodeoxynucleotide caused a slight reduction in the spheroid diameter to 90%, while the antisense oligonucleotide led to a rapid decrease in the spheroid diameter down to complete dissolution of the spheroid on the 12th day of incubation.

After co-incubation of the multicellular spheroids with oligonucleotides, these were furthermore tested in respect of their vitality with the aid of fluorescent dyes. The dyes used for this were fluorescein-labelled disodium acetate (FITC-FDA) and propidium iodide (PI). Each multicellular spheroid was incubated with 2 μl FITC-FDA in a concentration of 1 μmol/l for 20 minutes and with 10 μl PI (concentration: 20 μg/ml) for 10 minutes. Under a fluorescence microscope living cells appear green due to the FITC-FDA staining and dead cells appear red due to the PI staining. A pronounced cytotoxic reaction of the cells investigated in the antisense-treated group was found.

The results show that the antisense oligonucleotide according to the invention is cytotoxic to the tumour cell line tested and causes irreversible cell damage, which leads to death of the cell.

To rule out the solvent alone having an influence on growth, corresponding control experiments were carried out with the solvent (solvent; only the solvent of the oligonucleotides, but not the oligonucleotides themselves, was added), which showed that this influencing parameter was to be ignored (cf. FIG. 4).

### EXAMPLE 2

### Action on the Growth of RT4 Cells by Microinjection

The action of the oligonucleotides mentioned in example 1 on RT4 cells by direct injection of the compounds into the cell was investigated. The oligonucleotides were employed in non-modified (non-thiolated) form for this experiment. By this test, on the one hand the activity of non-modified oligonucleotides is to be demonstrated, and on the other hand non-specific binding of the oligodeoxynucleotides to cell membrane receptors being responsible for the effects described in example 1 is to be ruled out.

RT4 cells were sown on special cover glasses (CELLocate cover glasses, Eppendorf). A grid etched into the centre of these cover glasses facilitates finding the injected cells again. Before the cells were sown the cover glasses were placed in Petri dishes with a diameter of 3.5 cm and wetted with in each case 1 μl fibronectin, which ensures better attachment of the cells. 1.5×10⁵ cells, which had been dissolved beforehand by means of trypsin, were then sown per dish in 2.5 ml supplemented RPMI 1640 medium and were cultured at 37° C. overnight in an incubating cabinet.

The microinjection was carried out with the aid of a transjector 5246 and micromanipulator 5171 (Eppendorf) under light microscope control (inverse microscope type Leitz DMIL, Leica). The microinjection capillaries were filled with in each case 2.0 μl oligonucleotide solution (concentration 120 μmol/l) with the aid of Mikroloader® pipette tips (Eppendorf). The concentration was adjusted with sterile-filtered phosphate-buffered saline solution (PBS). To check the permeability of the filled capillaries, the clean function of the transjector was employed under microscopic control. With the capillary open, after immersion into the culture medium a uniform outflow of injection liquid was observed. The injection pressure was set empirically at 130 hPa and corrected after the first injections such that the injection led to a clear increase in the size of the cell, without destroying it. The injection time was between 0.3 and 0.5 second.

For the cytoplasmic injections, the capillary tip was brought up to the cytoplasm until a reflection of light caused by pressure on the cell was to be observed. The capillary was then raised again a few μm and the automatic injection movement was triggered by pressing the button. During the injection the injection limit could be corrected upwards or downwards in 0.14 μm steps, so that irregularities in the cell substrate could be compensated. For comparative studies, microinjection capillaries which were drawn in one working operation were used in order to keep the amount of liquid flowing out per injection as constant as possible for the same injection parameters. Nevertheless, the volume initiated varied from cell to cell, since the injection pressure and therefore the solution to be injected could spread out to a better or worse degree, depending on the region hit. To minimize the effects of cooling and a pH shift of the culture medium on the growth behaviour of the cells, the total injection time per cell culture dish was limited to 15 minutes.

The results of the test are plotted as a graph in FIG. 5. It was found that injection of antisense oligonucleotides and a subsequent incubation time of 22 hours resulted in a loss of adhesion in approx. 70% of the cells. Since only living cells remain adhered to the cover glass, this result is to be equated with death of 70% of the cells. Injection of the sense or missense oligonucleotides led only to a loss of adhesion in 30% of the cells in each case, and sole injection of the solvent (PBS) led to a loss of adhesion in 10% of the cells.

### EXAMPLE 3

### Action on the Growth of J82 Cells

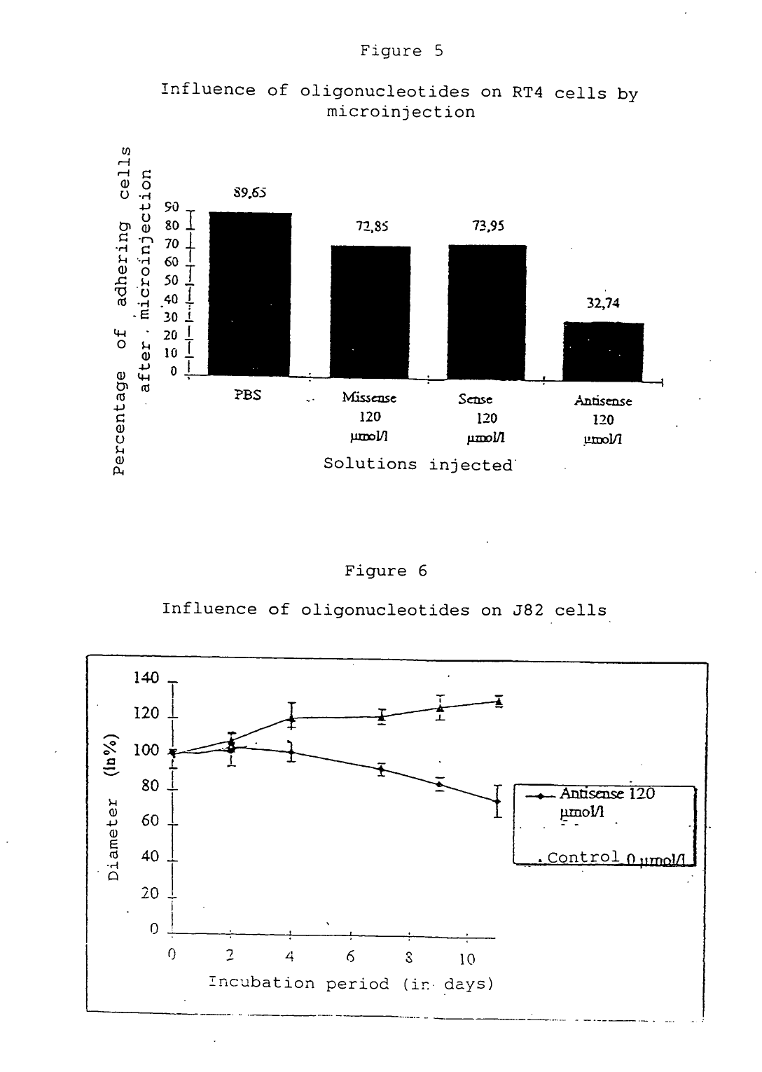

The action of the oligonucleotides on the human bladder tumour cells line J82 was investigated analogously to example 1. The thiolated antisense oligonucleotide in a concentration of 120 μmol/l led to a decrease in the spheroid diameter by 20% after 11 days, while the spheroid diameter of the control increased by about 30% in the same period of time (FIG. 6).

1. A method of preparing a medicament for destroying proliferation cells comprising:
combining an oligoribo- or oligodeoxyribonucleotide or a physiologically acceptable salt thereof which hybridizes with the mRNA which codes for the protein Ki-67 with a carrier to prepare a medicament for destroying proliferating cells.

2. A method according to claim 1, wherein the nucleotide sequence of the oligoribo- or oligodeoxyribonucleotide or physiologically acceptable salt thereof is complimentary to SEQ ID NO 1.

3. A method according to claim 2, wherein the nucleotide sequence of the oligoribo- or oligodeoxyribonucleotide or physiologically acceptable salt thereof is complimentary to the section from position 197 to 9962 of SEQ ID NO 1.

4. A method according to any one of claims 1 to 3, wherein the nucleotide sequence of the oligoribo- or oligodeoxyribonucleotide or physiologically acceptable salt thereof contains 12 to 66 nucleotides.

5. A method according to any one of claims 1-3, wherein the nucleotide sequence of the oligoribo- or oligodeoxyribonucleotide or physiologically acceptable salt thereof contains 17 to 46 nucleotides.

6. A method according to any one of claims 1-3, wherein the oligoribo- or oligodeoxyribonucleotide or physiologically acceptable salt thereof has the sequence (SEQ ID NO:3) (5′-ACC AGG CGT CTC GTG GGC CAC AT).

7. A method according to any one of claims 1-3, wherein one or more phosphate groups of the oligoribo- or oligodeoxyribonucleotide or physiologically acceptable salt thereof are replaced by at least one selected from the group consisting of phosphothioate, methylphosphonate, phosphoramidate, methylene (methylimino) and guanidine group(s).

8. A method according to any one of claims 1-3, wherein the oligoribo- or oligodeoxyribonucleotide or physiologically acceptable salt thereof has a terminal 3′-3′ and/or 5′-5′ internucleotide linkage.

9. A medicament comprising:
an oligoribo- and/or oligodeoxyribonucleotide or a physiologically acceptable salt thereof which is capable of hybridizing with the mRNA which codes for the cell cycle-associated protein Ki-67; and
conventional carrier substances, auxiliaries and/or additives, wherein the amount of oligonucleotide is adjusted such that an administration of 0.001 to 100 mg/kg of body weight is achieved.

10. A method according to any one of claims 1-3, wherein the medicament is formulated for treatment of tumours, autoimmune diseases, cicatrization, inflammations, allergies, rheumatic diseases and rejection reactions following transplantations.

11. An oligoribo- or oligodeoxyribonucleotide or a physiologically acceptable salt thereof containing 22 to 46 nucleotides and being capable of hybridizing with the mRNA which codes for the protein Ki-67.

12. An oligioribo- or oligodeoxyribonucleotide or physiologically acceptable salt thereof containing the sequence (SEQ ID NO:3) (5′-ACC AGG CGT CTC GTG GGC CAC AT) being capable of hybridizing with the mRNA which codes for the protein Ki-67.

13. A method according to claim 4, wherein the nucleotide sequence of the oligoribo- or oligodeoxyribonucleotide or physiologically acceptable salt thereof contains 17 to 46 nucleotides.

14. A method according to claim 4, wherein the oligoribo- or oligodeoxyribonucleotide or physiologically acceptable salt thereof has the sequence (SEQ ID NO:3) (5′-ACC AGG CGT CTC GTG GGC CAC AT).

15. A method according to claim 5, wherein the oligoribo- or oligodeoxyribonucleotide or physiologically acceptable salt thereof has the sequence (SEQ ID NO:3) (5′-ACC AGG CGT CTC GTG GGC CAC AT).

16. A method according to claim 4, wherein one or more phosphate groups of the oligoribo- or oligodeoxyribonucleotide or physiologically acceptable salt thereof are replaced by at least one selected from the group consisting of phosphothioate, methylphosphonate, phosphoramidate, methylene(methylimino) and guanidine group(s).

17. A method according to claim 5, wherein one or more phosphate groups of the oligoribo- or oligodeoxyribonucleotide or physiologically acceptable salt thereof are replaced by at least one selected from the group consisting of phosphothioate, methylphosphonate, phosphoramidate, methylene(methylimino) and guanidine group(s).

18. A method according to claim 6, wherein one or more phosphate groups of the oligoribo- or oligodeoxyribonucleotide or physiologically acceptable salt thereof are replaced by at least one selected from the group consisting of phosphothioate, methylphosphonate, phosphoramidate, methylene(methylimino) and guanidine group(s).

19. A method according to claim 4, wherein the oligoribo- or oligodeoxyribonucleotide or physiologically acceptable salt thereof has terminal 3′-3′ and/or 5′-5′ internucleotide linkage.

20. A method according to claim 5, wherein the oligoribo- or oligodeoxyribonucleotide or physiologically acceptable salt thereof has terminal 3′-3′ and/or 5′-5′ internucleotide linkage.

21. A method according to claim 6, wherein the oligoribo- or oligodeoxyribonucleotide or physiologically acceptable salt thereof has terminal 3′-3′ and/or 5′-5′ internucleotide linkage.

22. A method according to claim 7, wherein the oligoribo- or oligodeoxyribonucleotide or physiologically acceptable salt thereof has terminal 3′-3′ and/or 5′-5′ internucleotide linkage.

23. A method according to claim 4, wherein the medicament is formulated for treatment of tumours, autoimmune diseases, cicatrization, inflammations, allergies, rheumatic diseases or rejection reactions following transplantations.

24. A method according to claim 5, wherein the medicament is formulated for treatment of tumours, autoimmune diseases, cicatrization, inflammations, allergies, rheumatic diseases or rejection reactions following transplantations.

25. A method according to claim 6, wherein the medicament is formulated for treatment of tumours, autoimmune diseases, cicatrization, inflammations, allergies, rheumatic diseases or rejection reactions following transplantations.

26. A method according to claim 7, wherein the medicament is formulated for treatment of tumours, autoimmune diseases, cicatrization, inflammations, allergies, rheumatic diseases or rejection reactions following transplantations.

27. A method according to claim 8, wherein the medicament is formulated for treatment of tumours, autoimmune diseases, cicatrization, inflammations, allergies, rheumatic diseases or rejection reactions following transplantations.

28. An oligoribo- or oligodeoxyribonucleotide according to claim 11, characterized in that the nucleotide sequence of the oligoribo- or oligodeoxyribonucleotide or physiologically acceptable salt thereof is complementary to SEQ ID NO 1.

29. An oligoribo- or oligodeoxyribonucleotide according to claim 28, characterized in that the nucleotide sequence of the oligoribo- or oligodeoxyribonucleotide or physiologically acceptable salt thereof is complementary to the section from position 197 to 9962 of SEQ ID NO 1.

30. An oligoribo- or oligodeoxyribonucleotide according to claim 11, characterized in that one or more phosphate groups of the oligoribo- or oligodeoxyribonucleotide or physiologically acceptable salt thereof are replaced by phosphothioate, methylphosphonate, phosphoamidate, methylene (methylimino) and/or guanidine group(s).

31. An oligoribo- or oligodeoxyribonucleotide according to claim 12, characterized in that one or more phosphate groups of the oligoribo- or oligodeoxyribonucleotide or physiologically acceptable salt thereof are replaced by phosphothioate, methylphosphonate, phosphoamidate, methylene (methylimino) and/or guanidine group(s).

32. An oligoribo- or oligodeoxyribonucleotide according to claim 28, characterized in that one or more phosphate groups of the oligoribo- or oligodeoxyribonucleotide or physiologically acceptable salt thereof are replaced by phosphothioate, methylphosphonate, phosphoamidate, methylene (methylimino) and/or guanidine group(s).

33. An oligoribo- or oligodeoxyribonucleotide according to claim 29, characterized in that one or more phosphate groups of the oligoribo- or oligodeoxyribonucleotide or physiologically acceptable salt thereof are replaced by phosphothioate, methylphosphonate, phosphoamidate, methylene (methylimino) and/or guanidine group(s).

34. An oligioribo- or oligodeoxyribonucleotide according to claim 11, characterized in that the oligoribo- or oligodeoxyribonucleotide or physiologically acceptable salt thereof has a terminal 3′-3′ and/or 5′-5′ internucleotide linkage.

35. An oligoribo- or oligodeoxyribonucleotide according to claim 12, characterized in that the oligoribo- or oligodeoxyribonucleotide or physiologically acceptable salt thereof has a terminal 3′-3′ and/or 5′-5′ internucleotide linkage.

36. An oligoribo- or oligodeoxyribonucleotide according to claim 28, characterized in that the oligoribo- or oligodeoxyribonucleotide or physiologically acceptable salt thereof has a terminal 3′-3′ and/or 5′-5′ internucleotide linkage.

37. An oligoribo- or oligodeoxyribonucleotide according to claim 29, characterized in that the oligoribo- or oligodeoxyribonucleotide or physiologically acceptable salt thereof has a terminal 3′-3′ and/or 5′-5′ internucleotide linkage.

38. An oligoribo- or oligodeoxyribonucleotide according to claim 32, characterized in that the oligoribo- or oligodeoxyribonucleotide or physiologically acceptable salt thereof has a terminal 3′-3′ and/or 5′-5′ internucleotide linkage.

39. An oligoribo- or oligodeoxyribonucleotide according to claim 33, characterized in that the oligoribo- or oligodeoxyribonucleotide or physiologically acceptable salt thereof has a terminal 3′-3′ and/or 5′-5′ internucleotide linkage.

40. A method according to claim 1, wherein the carrier is selected to provide a medicament for application systemically, locally, subcutaneously, intrathecally, topically or by enema.

41. A method according to claim 1, wherein the carrier is selected to provide a medicament in which the oligoribo- or oligodeoxyribonucleotide or physiologically acceptable salt thereof is present in solution, emulsion or solid form.

42. A method according to claim 1, further comprising the step of adding an auxiliary or additive.

43. A method according to claim 1, wherein the oligoribo- or oligodeoxyribonucleotide or physiologically acceptable salt thereof hybridizes with Ki-67 at 37° C.

44. A method according to claim 1, wherein the nucleotide sequence of the oligoribo- or oligodeoxyribonucleotide or physiologically acceptable salt thereof is complimentary to the section from position 197 to 220 of SEQ ID NO 1.

45. A method according to claim 1, wherein the nucleotide sequence of the oligoribo- or oligodeoxyribonucleotide or physiologically acceptable salt thereof is complimentary to the section from position 2673 to 9962 of SEQ ID NO 1.

46. A method according to claim 7, wherein all of the phosphate groups are replaced with at least one selected from the group consisting of phosphothioate, methylphosphonate, phosphoramidate, methylene (methylimino) and guanidine group(s).

47. A method according to claim 7, wherein one or more of the phosphate groups are replaced with phosphothioate.

48. A method according to claim 1, further comprising the step of modifying at least one of bases, sugar residues or phosphate residues of the oligoribo- or oligodeoxyribonucleotide or physiologically acceptable salt thereof to improve ability to penetrate through membranes and/or to increase a biological half-life.

49. A method according to claim 21, further comprising replacing one or more ribose residues of the oligoribo- or oligodeoxyribonucleotide or physiologically acceptable salt thereof with hexose or an amino acid.

50. A method according to claim 21, further comprising modifying one or more bases of the oligoribo- or oligodeoxyribonucleotide or physiologically acceptable salt thereof with 5-propinyl-uracyl, 5-propinylcytosine and/or tricyclic cytosine analogue phenoxazine.

51. A method according to claim 1, wherein medicament is prepared such that administration of the medicament provides 0.001 to 100 mg of the oligoribo- or oligodeoxyribonucleotide or a physiologically acceptable salt thereof per kilogram of body weight.

52. A method according to claim 1, wherein medicament is prepared such that administration of the medicament provides 0.001 to 10 mg of the oligoribo- or oligodeoxyribonucleotide or a physiologically acceptable salt thereof per kilogram of body weight.

53. A method according to claim 1, wherein medicament is prepared such that administration of the medicament provides 0.001 to 3 mg of the oligoribo- or oligodeoxyribonucleotide or a physiologically acceptable salt thereof per kilogram of body weight.

54. A medicament comprising:
at least one oligoribo- or oligodeoxyribonucleotide or a physiologically acceptable salt thereof which hybridizes with the mRNA which codes for the protein Ki-67; and
a carrier.

55. A medicament according to claim 54, wherein the nucleotide sequence of the oligoribo- or oligodeoxyribonucleotide or physiologically acceptable salt thereof is complimentary to SEQ ID NO 1.

56. A medicament according to claim 55, wherein the nucleotide sequence of the oligoribo- or oligodeoxyribonucleotide or physiologically acceptable salt thereof is complimentary to the section from position 197 to 9962 of SEQ ID NO 1.

57. A medicament according to claim 54, wherein the nucleotide sequence of the oligoribo- or oligodeoxyribonucleotide or physiologically acceptable salt thereof contains 12 to 66 nucleotides.

58. A medicament according to claim 54, wherein the nucleotide sequence of the oligoribo- or oligodeoxyribonucleotide or physiologically acceptable salt thereof contains 17 to 46 nucleotides.

59. A medicament according to claim 54, wherein the oligoribo- or oligodeoxyribonucleotide or physiologically acceptable salt thereof contains the sequence (SEQ ID NO:3) (5′-ACC AGG CGT CTC GTG GGC CAC AT).

60. A medicament according to claim 54, wherein one or more phosphate groups of the oligoribo- or oligodeoxyribonucleotide or physiologically acceptable salt thereof are replaced by at least one selected from the group consisting of phosphothioate, methylphosphonate, phosphoramidate, methylene (methylimino) and guanidine group(s).

61. A medicament according to claim 54, wherein the oligoribo- or oligodeoxyribonucleotide or physiologically acceptable salt thereof has a terminal 3′-3′ and/or 5′-5′ internucleotide linkage.

62. A medicament according to claim 54, wherein the medicament is formulated for treatment of tumours, autoimmune diseases, cicatrization, inflammations, allergies, rheumatic diseases or rejection reactions following transplantations.

63. (Cancelled)

64. A medicament according to claim 54, wherein the medicament is formulated for systemic application.

65. A medicament according to claim 54, wherein the medicament is formulated for local application.

66. A medicament Medicamont according to claim 54, wherein the medicament is formulated for subcutaneous application.

67. A medicament according to claim 54, wherein the medicament is formulated for intrathecal application.

68. A medicament according to claim 54, wherein the medicament is formulated for topical application.

69. A medicament according to claim 54, wherein the medicament is formulated for application by enema.

70. A medicament according to claim 54, wherein the oligoribo- or oligodeoxyribonucleotide or physiologically acceptable salt thereof is present in solution, emulsion or solid form.

71. A medicament according to claim 54, further comprising an auxiliary or additive.

72. A medicament according to claim 54, wherein the oligoribo- or oligodeoxyribonucleotide or physiologically acceptable salt thereof hybridizes with Ki-67 at 37° C.

73. A medicament according to claim 54, wherein the nucleotide sequence of the oligoribo- or oligodeoxyribonucleotide or physiologically acceptable salt thereof is complimentary to the section from position 197 to 220 of SEQ ID NO 1.

74. A medicament according to claim 54, wherein the nucleotide sequence of the oligoribo- or oligodeoxyribonucleotide or physiologically acceptable salt thereof is complimentary to the section from position 2673 to 9962 of SEQ ID NO 1.

75. A medicament according to claim 54, wherein all of the phosphate groups are replaced with at least one selected from the group consisting of phosphothioate, methylphosphonate, phosphoramidate, methylene (methylimino) and guanidine group(s).

76. A medicament according to claim 60, wherein one or more of the phosphate groups are replaced with phosphothioate.

77. A medicament according to claim 54, wherein at least one of bases, sugar residues or phosphate residues of the oligoribo- or oligodeoxyribonucleotide or physiologically acceptable salt thereof has been modified to improve ability to penetrate through membranes and/or to increase a biological half-life.

78. A medicament according to claim 54, wherein one or more ribose residues of the oligoribo- or oligodeoxyribonucleotide or physiologically acceptable salt thereof have been replaced with hexose or an amino acid.

79. A medicament according to claim 54, wherein one or more bases of the oligoribo- or oligodeoxyribonucleotide or physiologically acceptable salt thereof have been modified with 5-propinyl-uracyl, 5-propinylcytosine and/or tricyclic cytosine analogue phenoxazine.

80. A medicament according to claim 54, wherein the medicament provides 0.001 to 100 mg of the oligoribo- or oligodeoxyribonucleotide or a physiologically acceptable salt thereof per kilogram of body weight when administered.

81. A medicament according to claim 54, wherein the medicament provides 0.001 to 10 mg of the oligoribo- or oligodeoxyribonucleotide or a physiologically acceptable salt thereof per kilogram of body weight when administered.

82. A medicament according to claim 54, wherein the medicament provides 0.001 to 3 mg of the oligoribo- or oligodeoxyribonucleotide or a physiologically acceptable salt thereof per kilogram of body weight when administered.

83. A method according to claim 1, wherein the nucleotide sequence of the oligoribo- or oligodeoxyribonucleotide or physiologically acceptable salt thereof is complimentary to the section from position 197 to 2673 of SEQ ID NO 1.

84. A medicament according to claim 54, wherein the nucleotide sequence of the oligoribo- or oligodeoxyribonucleotide or physiologically acceptable salt thereof is complimentary to the section from position 197 to 2673 of SEQ ID NO 1.
